(19) United States
(12) Patent Application Publication
(10) Pub. No.: US 2015/0111852 A1
(43) Pub. Date: Apr. 23, 2015

(54) 1-Di(sec-butyl)-phosphinoyl-pentane (dapa-2-5) as a topical agent...

(71) Applicant: Edward T. Wei, Berkeley, CA (US)

(72) Inventor: Edward T. Wei, Berkeley, CA (US)

(21) Appl. No.: 14/544,042

(22) Filed: Nov. 18, 2014

Related U.S. Application Data
(63) Continuation-in-part of application No. PCT/GB2013/052751, filed on Oct. 22, 2013.

(30) Foreign Application Priority Data
Oct. 22, 2013 (GB) PCT/GB2013/052751

(51) Int. Cl. A61K 31/66

(52) U.S. Cl. CPC A61K 31/66
USPC 514/75

(57) ABSTRACT

The concept is put forward here that heat abstraction sensations, captured by topical application of a molecule, can be used to alleviate discomfort of non-keratinizng tissues. By synthesizing compounds and devising tests, a molecule name DAPA-2-5 was identified as having the selective desirable properties for achieving this purpose. DAPA-2-5 is a di-alkyl-phosphinoyl-alkane, and “DAPA-2-5” is 1-Di(sec-butyl)-phosphinoyl-pentane. DAPA-2-5 evokes a dynamic cooling sensation on non-keratinizing body surfaces (including, e.g., oropharyngeal, esophageal, and anogenital surfaces) which is not accompanied by stinging, irritation, or unpleasant tastes. Thus, it can be used to treat (e.g., suppress) sensory discomfort from non-keratinizing stratified epithelium (NKSE) selectively. This unusual selectivity, potency, and efficacy was also surprisingly exhibited in laboratory animal tests of inhibition of heat-induced edema, of eliciting skin irritation, and of inhibition of acid-induced swallowing. DAPA-2-5 is useful, for example, in the treatment of disorders (e.g., diseases) including sensory discomfort from non-keratinizng stratified epithelial (NKSE) tissue; upper aerodigestive tract discomfort; oropharyngeal discomfort; esophageal discomfort; throat irritation; cough; heartburn; chest pain; anogenital discomfort; or inflammation of non-keratinizng stratified epithelial (NKSE) tissue. The present discovery also pertains to pharmaceutical compositions comprising DAPA-2-5 and DAPA compositions, for example, in therapy and in differential diagnosis. A particularly favoured embodiment is 1-(Di-sec-butyl-phosphinoyl-pentane as an orally disintegrating tablet formulated with a mineral excipient called magnesium aluminosilicate.

### TECHNICAL FIELD

The present discovery pertains generally to the field of therapeutic compounds. More specifically the present discovery pertains to a particular di-alkyl-phosphinoyl-alkane, 1-Di(sec-butyl)-phosphinoyl-pentane, referred to herein as “DAPA-2-5”. Surprisingly and unexpectedly, DAPA-2-5, is able to treat (e.g., suppress) sensory discomfort from non-keratinizng stratified epithelium (NKSE) selectively, that is, without the problems of stinging, irritancy, or unpleasant tastes, for example, as found with structurally similar compounds. As described herein, DAPA-2-5 is able to evoke a dynamic cooling sensation on non-keratinizng body surfaces (including, e.g., oropharyngeal, esophageal, and anogenital surfaces) which is not accompanied by stinging or other unpleasant sensations. Consequently, DAPA-2-5 is useful, for example, in the treatment of disorders (e.g., diseases) including sensory discomfort from non-keratinizng stratified epithelial (NKSE) tissue; upper aerodigestive tract discomfort; oropharyngeal discomfort; esophageal discomfort; throat irritation; cough; heartburn; chest pain; anogenital discomfort; or inflammation of non-keratinizng stratified epithelial (NKSE) tissue. The present discovery also pertains to pharmaceutical compositions comprising DAPA-2-5, and the use of DAPA-2-5 and DAPA-2-5 compositions, for example, in therapy and as an agent for differential diagnosis.

### BACKGROUND

A number of publications are cited herein in order to more fully describe and disclose the discovery and the state of the art to which the discovery pertains. Each of these publications is incorporated herein by reference in its entirety into the present disclosure, to the same extent as if each individual publication was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present discovery. It is not an admission that any of the information provided herein is prior art or relevant to the presently claimed invention, or that any publication specifically or implicitly referenced is prior art.

Thermal Comfort, Sensory Discomfort, and Nociception

“Thermal comfort” is a well-developed concept in ergonomics, the apparel industry, engineering, and architecture. The term refers to a condition in which a person wearing a normal amount of clothing feels neither too cold nor too warm. Thermal comfort is important for one's well-being and for productivity. It is achieved when the air temperature, humidity, and air movement are within a specified range called the “comfort zone” [24° C. to 27° C. for UK and USA]. Standardized descriptors of thermal comfort were developed by Bedford and ASHRAE [American Society of Heating, Refrigeration, and Air-conditioning Engineers]. The table of cooling scale is taken from Auliciems and Szokolay: Thermal Comfort, 2007.as shown in the Table below. I have added the number −4 on the sensory scale to describe frigid or stinging, icy cold or “Arctic cold.”

TABLE 1
Verbal Descriptors of Thermal Comfort

| Numerical Value | ASHRAE | Bedford |
|---|---|---|
| 3 | Hot | Much too warm |
| 2 | Warm | Too warm |
| 1 | Slightly warm | Comfortable warm |
| 0 | Neutral | Comfortable |
| −1 | Slightly cool | Comfortably cool |
| −2 | Cool | Too cool |
| −3 | Cold | Much too cool |
| −4 | “Arctic cold”, frigid | Stinging, pain |

The concept of thermal comfort was developed for conditions of the skin which has a keratin layer and blood vessels innervated by sympathetic nerves that will constrict in response to cold. The skin temperatures of the feet and hands, for example, will drop to 25° C. and 30° C., respectively, when the ambient temperature is 23° C. but the rectal and oral temperatures remains constant at 36° C. to 37° C. from ambient 23° C. to 34° C. For non-keratinizng tissues, such as the oral cavity, pharyngeal, or esophageal or rectal mucosae, the internal blood flow provides heat and the tissue temperatures are kept constant. Hence it is difficult to sustain heat abstraction sensations with physical methods, such as ice or cold liquids, in these tissues.

Nociception may be defined as the neural encoding and processing of noxious stimuli. Of particular interest here are anti-nociceptive drugs that act peripherally and can be applied topically. By “anti-nociceptive”, it is meant that the drug suppresses the psychical and physiological perception of and reaction to the noxious stimuli. By “peripherally”, it is meant that the primary site of the drug action is located outside the central nervous system; that is, outside of the brain and spinal cord.

There are currently two major classes of anti-nociceptive drugs that act peripherally to attenuate transmission of nociceptive (noxious) signals to the central nervous system. One class is local anaesthetics, such as procaine and lidocaine, which act on sodium channels of peripheral nerve fibres to inhibit nerve conduction of nociceptive signals towards the central nervous system. Another class is agents, such as aspirin and ibuprofen, which inhibit the synthesis of certain prostaglandins. These prostaglandins, when released by tissues during injury or inflammation, lower the threshold for firing of sensory nerve fibres that respond to noxious stimuli. Yet another class of anti-nociceptive drugs is the narcotic analgesics, which do not suppress pain via peripheral actions but instead act directly on neuronal elements in the brain and spinal cord.

Pain, defined by Sir Charles Sherrington as “the psychical adjunct of an imperative protective reflex”, is activated by increased discharge of unmyelinated small-diameter sensory fibres called polymodal C fibres. Pain is categorized as nociceptive or neuropathic. Nociceptive pain is caused by cell injury and neuropathic pain is caused by damage to the nerve fibres that transmit the pain signals. There are many conditions that produce pain; the most common being, for example, trauma, inflammation, and immune disorders. Sensations that may accompany pain are irritation, pruritus (itch), burning sensations (dysesthesias) and a sense of malaise and disaffection. As used herein, the psychical adjuncts of nociception are together categorized as “sensory discomfort”.

There are four basic types of animal tissues: connective tissue, muscle tissue, nervous tissue and epithelial tissue. Epithelial cells line ducts, cavities and surfaces of organs throughout the body. When the layer is one cell thick, it is called simple epithelium. If there are two or more layers of cells, it is called stratified epithelium. Stratified epithelium is composed mainly of squamous (flattened) cells and some cuboidal cells. Historically, stratified epithelia were divided into two broad categories: keratinized stratified epithelia, and non-keratinized stratified epithelia. Keratinized epithelium, such as the epidermis of the skin, has an exterior layer of dead cells [stratum corneum] composed of keratin proteins that are tough and water-impermeable. Keratin also covers the filiform papillae of the tongue. By contrast, non-keratinizing stratified epithelia do not contain a significant keratin layer and are present principally on: the lining of the nasal cavity; portions of the oral cavity such as the internal lips; the pharyngeal surface; the esophageal surface; the lining of the respiratory tree; and the anogenital surface. The term “cornified epithelia” has traditionally been reserved for the keratin covered tissues such as nails, hair and hooves.

Keratinizing tissues withstand injury better than non-keratinizing tissues. Non-keratinizing epithelial surfaces must be kept moist by glandular (serous and mucous) secretions in order to avoid desiccation. A layer of keratin is a formidable barrier for drug access to neuronal receptive fields embedded in tissues underneath the keratin. Non-keratinizing epithelial surfaces are more sensitive to sensory agents because this barrier is absent or diminished.

In recent years, it is recognized that keratin is a family of proteins and keratin filaments form an integral part of the cytoskeleton of all epithelial cells. Hence, the term “non-keratinized stratified epithelia” is no longer accurate and may eventually become obsolete. The preferred term at the moment is “non-keratinizing stratified epithelia” implying that these cells do not form an external keratin layer. Bragulla and Homberger [Structure and functions of keratin proteins in simple, stratified, keratinized and cornified epithelia. Journal of Anatomy, 214: 516-59, 2009] have proposed to divide stratified epithelia into “cornified epithelia” and “keratinized epithelia”, but it is not yet clear if such new terminology is accepted.

In this application, the term “keratinizing stratified epithelia” and “non-keratinizing stratified epithelia” will be used to distinguish between the two categories of tissues. The term “non-keratinized stratified epithelia” is used in the title of this application to maintain consistency with traditional nomenclature.

There are currently no topical anti-nociceptive (pain-suppressant) compounds that have strong efficacy on sensory discomfort from non-keratinizng stratified epithelium (NKSE). This is especially true for sensory discomfort from the oral cavity, pharyngeal, and oesophageal surfaces.

Local anaesthetic compounds such as lidocaine are sometimes used for pain and discomfort from anogenital surfaces (e.g., for vulvovaginal pain) and from the pharynx (e.g., for pharyngitis) but these drugs can cause hypersensitivity reactions and have the undesirable property of numbing the tissues to touch and pressure. Prolonged use is dangerous because this class of drugs inhibits epithelial cell growth.

The non-steroidal anti-inflammatory compounds (NSAIDs), for example, ketorolac do not work for pain arising from anogenital or oral cavity NKSE. Anti-inflammatory steroids, by reducing inflammation, can reduce nociception, but the onset of anti-nociceptive action is not immediate.

Menthol has some limited analgesic action in ointments for hemorrhoidal discomfort. In lozenges and confectionery, menthol has some benefit for sore or irritated throats and for cough. On keratinized skin, high concentrations of menthol (for example, more than 2% by weight) can be applied without direct irritation to the skin. For example, topical patches containing 5% by weight menthol (e.g., IcyHot Medicated Patch; Chattem, Inc.) can be applied onto the skin of the torso to relieve muscular pain. On non-keratinizng epithelia, however, the irritating effects of menthol limit its use: in the oral cavity, for example, lozenges containing more than 8 mg of menthol per unit are aversive in taste; menthol irritates the nasal membranes at higher concentrations and inhaled menthol will increase nasal secretions.

A number of menthol-related compounds with physiological cooling effects on keratinized epithelia such as the skin and the tongue have been described by Watson et al. [“Compounds with the Menthol Cooling Effect”, J. Soc. Cosmet. Chem. 29: 185-200, 1978]. Trialkylphosphine oxides having a “physiological cooling action” were described [Rowsell et al. “Phosphine oxides having a physiological cooling effect”, U.S. Pat. No. 4,070,496, 1978]. The studies described therein relate to compounds that affect sensory processes on keratinized surfaces because most of the testing was done on the tongue and the tongue is keratinized. But the anti-nociceptive actions of these molecules on non-keratinizng tissues were not clearly recognized or systemically investigated.

There is a need for a new class of pharmacological agent that can selectively suppress sensory discomfort arising from non-keratinizng stratified epithelium (NKSE), but without problems of aversive tastes, irritancy, or toxicity.

If one considers sensations arising from NKSE, there is an obvious qualitative difference from that of the keratinized skin. Note, for example, the sting and pain from the eye after exposure to ethanol fumes; the reaction of the nasal membranes to water; the choking sensations of chili pepper in the throat; and the sour, acrid taste of regurgitated acid in the mouth. These sensations are clearly different from what can be felt on the skin. Here, the nerve endings that report noxious signals from NKSE iginate mostly from cranial nerves such as the trigeminal (5^{th}), glossopharyngeal lossopharyngeal (9^{th}), and vagal nerves (10^{th}), and from some spinal sensory afferents of the NKSE, but not from the skin or tongue. As discussed herein, the effects of agents designed for alleviating sensory discomfort from NKSE clearly differ from the effects of agents designed for keratinized skin.

Wei has previously described two compounds (both p-menthane carboxamide amino acid ester derivatives) that are selectively cooling on the eye surfaces relative to cooling on the keratinized philtrum skin [Wei, Sensory /cooling agents for skin discomfort. Journal Skin Barrier Research. 14: 5-12, 2012, and US 084226463, Apr. 23, 2013]. These observations provide the first evidence that selective anti-nociception can be achieved on NKSE. These compounds produce sensations of heat abstraction which on the ASHRAE scale correspond to −1 [mild cool], −2 [cool], and −3 [cold].

“Comfort” is the result of optimized sensory inputs, and the concept of thermal comfort for keratinized skin can also be applied to non-keratinizng tissues. In inflammation the cardinal signs are a feeling of heat [calor], redness [rubor], and pain [dolor] in the injured tissues and it may be expected that sensations of heat abstraction will relieve sensory discomfort. Thus, a chemical that simulates the sensations of heat abstraction can be used, without a decrease in tissue temperatues, to produce “comfort” or to relieve discomfort.

Further refinement on the design of molecules that have the characteristic desired sensation necessary to obtain comfort or relieve discomfort on NKSE is described herein. In particular, if a candidate compound elicits “dynamic cool” on a NKSE surface without irritancy, that is, a ASHRAE sensation between −2 and −3, then it is a good candidate to treat sensory discomfort from that tissue. An exceptional compound with the desired qualities and selected for this use is DAPA-2-5.

Known Phosphine Oxides

The 1-dialkyl-phosphinoyl-alkanes [total number of carbons 15] are solvent-like molecules that require only several [1 to 3] steps for synthesis. They are also known as trialkylphosphine oxides, but the preferred term now is dialkyl-phosphinoyl-alkane [abbreviated here as DAPA].

Rowsell et al., 1978, describes a range of phosphine oxides which have a physiological cooling effect on skin and on the mucous membranes of the body, particularly the nose, mouth, throat and gastrointestinal tract. See, e.g., the table in columns 3 and 4 therein. The 18th entry in that table is 1-Di(sec-butyl)-phosphinoyl-pentane, referred to herein as DAPA-2-5.

Wei [Ophthalmic compositions and method for treating eye discomfort and pain US patent publication number 2005/0059639 A1, 2005] describes the use of certain phosphine oxides and the treatment of eye discomfort by the adminstration of eye drops containing those compounds in an ophthalmic solution. See, e.g., Table 1 on page 4 therein. The 13th entry in that table is 1-Di(sec-butyl)-phosphinoyl-pentane, referred to herein as DAPA-2-5.

### SUMMARY OF THE DISCOVERY

The present discovery pertains to a particular di-alkyl-phosphinoyl-alkane, 1-Di(sec-butyl)-phosphinoyl-pentane, referred to herein as “DAPA-2-5”. Surprisingly and unexpectedly, DAPA-2-5, is able to treat (e.g., suppress) sensory discomfort from non-keratinizng stratified epithelium (NKSE) selectively, that is, without the problems of stinging, irritancy, or bad taste, for example, as found with structurally similar compounds.

As described herein, DAPA-2-5 is able to evoke a dynamic cooling sensation on non-keratinizng body surfaces (including, e.g., oropharyngeal, esophageal, and anogenital surfaces) which is not accompanied by stinging or other unpleasant sensations. Consequently, DAPA-2-5 is useful, for example, in the treatment of disorders (e.g., diseases) including sensory discomfort from non-keratinizng stratified epithelial (NKSE) tissue; upper aerodigestive tract discomfort; oropharyngeal discomfort; esophageal discomfort; throat irritation; cough; heartburn; chest pain; anogenital discomfort; or inflammation of non-keratinizng stratified epithelial (NKSE) tissue. Furthermore, it may be used as a differential diagnostic agent for non-cardiac versus cardiac pain. The present discovery also pertains to pharmaceutical compositions comprising DAPA-2-5, especially as formulations with mineral excipients, and the use of DAPA-2-5 and DAPA-2-5 compositions, for example, in therapy.

One aspect of the present discovery pertains to a particular di-alkyl-phosphinoyl-alkane, 1-Di(sec-butyl)-phosphinoyl-pentane, referred to herein as “DAPA-2-5”, for use in a method of treatment (e.g., selective treatment) of certain disorders (e.g., diseases), as described herein.

Another aspect of the present discovery pertains to use of DAPA-2-5 in the manufacture of a medicament for treatment (e.g. selective treatment of diseases), as described herein.

Another aspect of the present discovery pertains to a method of treatment (e.g., selective treatment) of certain disorders (e.g., diseases), as described herein, comprising administering to a patient in need of treatment a therapeutically effective amount of DAPA-2-5, preferably in the form of a pharmaceutical composition.

Another aspect of the present discovery pertains to a composition for formulations for topical delivery to the upper digestive tract comprising (a) DAPA compounds, as described herein, preferably provided as a pharmaceutical composition mixed with an inorganic mineral excipient, and in a suitable compressed state of an orally dissolving tablet.

Another aspect of the present discovery pertains to a kit comprising (a) DAPA-2-5, as described herein, as a tool for the differential diagnosis of non-cardiac versus cardiac pain, and (b) instructions for use, for example, written instructions on how to administer and use the compound.

Another aspect of the present discovery pertains to a kit comprising (a) DAPA-2-5, as described herein, preferably provided as a pharmaceutical composition and in a suitable container and/or with suitable packaging; and (b) instructions for use, for example, written instructions on how to administer the compound.

As will be appreciated by one of skill in the art, features and preferred embodiments of one aspect of the discovery will also pertain to other aspects of the discovery.

### BRIEF DESCRIPTION OF THE DRAWINGS

FIG. 1 is a graph of fluorescence (Relative Fluourescence Units; % Maximum) of test compounds, evoked from cells transfected with TRPM8 plasmid, as a function of the logarithm of the concentration of the test compound (μM), for each of DAPA-2-4 (circle), DAPA-2-5 (square), DAPA-2-6 (inverted triangle), DAPA-2-7 (diamond), and DAPA-2-8 (star).

FIG. 2 shows the relative activity of DAPA compounds for the inhibition of heat-induced edema in the anesthetized rat paw. DAPA-2-5 exposure significantly inhibited heat-induced edema relative to the contralateral paw by 12.9% (P<0.01). This effect was not seen with the other DAPA compounds tested and indicated that DAPA-2-5 had anti-inflammatory properties.

FIG. 3 shows graphs of the amplitude (mV) of electromyogram (EMG) activity recorded from the myohyloid muscle of the anesthetized rat after infusion of 0.1 N HCl into the oropharynx. Swallowing movements are provoked by the acid irritant. These responses are inhibited by prior infusion of DAPA-2-5. FIG. 3A: 47 swallows after acid. FIG. 3B: DAPA-2-5, 0.4 mg/mL reduces the acid response to 3 swallows within 3 min. FIG. 3C: inhibition persists 8 min after DAPA-2-5, with 9 swallows after acid challenge. FIG. 3D: gradual recovery of response at 22 min after DAPA-2-5, with 27 swallows.

FIG. 4 shows a comparison of the relative potency of each DAPA compound for inhibition of acid-induced swallowing [IC₅₀] and for TRPM8-receptor activation [EC₅₀]. The relative potency is proportional to the reciprocal of the IC₅₀ and EC₅₀ values.

FIG. 5 shows polarization traces that illustrate, in the first trace, FIG. 5A (“Wild Type”), the inhibition of capsaicin-induced depolarization of the isolated mouse vagus by DAPA-2-5, superfused at 1 mg/mL, and, in the second trace, FIG. 5B (“TRPM8 KO”), the significant absence of inhibition in the isolated TRPM8 KO mouse vagus by DAPA-2-5, superfused at a 1 mg/mL.

FIG. 6 shows cough frequency of guinea pigs exposed to citric acid mist. Guinea pigs (N=20) exposed to 1 M citric acid solution had a cough frequency of 19.9±3.9 coughs per 10 min observation period. One week later, saline delivered into the oropharyngeal region did not influence cough frequency (18.0±4.0 coughs), but DAPA-2-5, 2 mg/mL in saline, delivered at 75 μL per animal, inhibited cough (6.7±1.6 coughs, P<0.01 Mann-Whitney U test).

### DETAILED DESCRIPTION OF THE DISCOVERY

Menthol is used on the skin (e.g., Icy-Cold Patches, Ben-Gay Ointment), in the mouth (e.g., in candy and lozenges), and in the nose (e.g., Vick's vapo-inhaler) to relieve sensory discomfort, but its actions on surfaces without a tough keratin cover (i.e., tissues other than the skin and tongue) are limited by irritant effects and by a limited time of action. About three decades ago, Watson et al. synthesized over 1200 compounds in an attempt to find cooling agents that had properties better than menthol [New compounds with the menthol cooling effect. J. Soc. Cosmet. Chem. 29: 185-200, 1978]. From this research, an N-alkyl-cycloalkyl- and an N-alkyl-alkyl carboxamide, WS-3, WS-5, and WS-23, were marketed and today used as additives for confectionery, comestibles, (e.g., chewing gum), and toiletries. These scientists also described phosphine oxides [Rowsell et al., 1978] with cooling properties. However, the phosphine oxides were not developed or commercialized.

Watson et al. [US Patent No. 4,178,459] tested the properties of cooling agents, including phosphine oxides, on volunteers by putting filter paper (1×1 cm), impregnated with a known amount of compound, onto the dorsal surface of the tongue of the test subject. After 30 sec, the subject was required to report presence or absence of a cooling effect. These data were reported as “Threshold, pg” and refer to the threshold amount of the test substance that produces cooling sensations upon application onto the tongue of a panel of human volunteers. The average threshold of (-)-menthol for 6 subjects was 0.25 μg, but there was a 100-fold variation in individual sensitivity. The surface of the tongue is keratinized and relatively insensitive to thermosensation. Moreover, coolness signals detected from the dorsal surface of the tongue may be confounded by gustatory, olfactory, and other variables.

As described herein, the Inventor has re-examined the known phosphine oxide compounds with the goal of finding an optimal candidate to soothe non-keratiniznq tissues, such as surfaces of the mouth, throat, esophagus, and anogenitalia, but without irritant or undesirable characteristics. The goal was to find new chemical entities that can be used as single agents for pharmaceuticals, and not another agent for comestibles or toiletries. Therefore, the screening methods, bioassays, and animal models are aimed at pharmaceutical endpoints and applications, and at target surfaces that are non-keratinizng.

Surprisingly and unexpectedly, one compound, referred to herein as DAPA-2-5, was found to have an ideal combination of properties. As described in the studies below:
DAPA-2-5 evokes a dynamic cooling sensation on NKSE without cold discomfort (Study 3), an effect not seen with 10 other analogs.
DAPA-2-5′s dynamic cooling sensations are sufficiently prolonged [10 to 15 min] and leaves a residual anti-nociceptive effect to be of therapeutic benefit.
DAPA-2-5 suppresses the irritative effect of a chili-pepper sauce on the throat of volunteer subjects (Study 4).
The unusual properties of DAPA-2-5 could not have been predicted based on its TRPM8 receptor activation potency (Study 5), but had to be discovered by experiment. There is no direct correlation between the EC₅₀ [measurement of TRPM8 potency] and efficacy for anti-nociception on NKSE among a large number of tested analogs.
DAPA-2-5 selectively suppresses heat-induced edema in an anesthetized animal model (Study 6) and, by itself, does not stimulate inflammation (Study 6), an effect not seen with structurally related analogs.
DAPA-2-5 potently and selectively suppresses acid-induced swallowing in an anesthetized animal model (Study 8), an effect that clearly distinguishes it from other structurally related analogs.
DAPA-2-5 inhibits capsaicin-induced depolarization in the isolated mouse vagus (Study 9) and cough frequency in a citric acid acid-induced model of cough in the guinea pig. (Study 10).

These results, in multiple test systems, show that DAPA-2-5 exhibits an unusual selective drug action. Consequently, DAPA-2-5 is selectively useful, for example, in the treatment of disorders (e.g., diseases) of sensory discomfort from non-keratinizng stratified epithelial (NKSE) tissue; upper aerodigestive tract discomfort; oropharyngeal discomfort; esophageal discomfort; throat irritation; cough; heartburn; chest pain; anogenital discomfort; or inflammation of non-keratinizng stratified epithelial (NKSE) tissue.

DAPA Compounds

The discovery relates to a particular compound which is an example of the group of compounds known as phosphine oxides (which have the following general formula), and more particularly, an example of the group known as di-alkyl-phosphinoyl-alkanes (herein referred to as “DAPA compounds”) (wherein each of R₁, R₂, and R₃ is an alkyl group).

(O=)P R₁ R₂ R₃

More specifically, the discovery relates to a particular di-alkyl-phosphinoyl-alkane, 1-Di(sec-butyl)-phosphinoyl-pentane, referred to herein as “DAPA-2-5”.

TABLE 2
Chemical structure of DAPA-2-5

| | Chemical | Formula/ | |
|---|---|---|---|
| Code | Name | Weight | Chemical Structure |
| DAPA- 2-5 | 1-Di(sec- butyl)- phos- phinoyl- pentane | C₁₃H₂₉OP 232.35 | 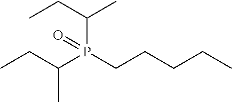 |

DAPA-2-5 is a liquid at room temperature, with a density of ˜0.85 g/cm³ and a boiling point of 112-120° C.

Note that each of the sec-butyl groups of DAPA-2-5 has a chiral centre, and that each chiral centre may independently be in the (R) or (S) configuration. As a consequence, DAPA-2-5 has four possible stereoisomers: two optically active stereoisomers (i.e., R,R and S,S), and two optically inactive meso forms (i.e., R,S and S,R). Unless otherwise indicated, a reference to DAPA-2-5 is intended to be reference to any one of the four stereoisomers, and any mixture of any two or more of the four stereoisomers.

Following extensive studies, the Inventor has identified DAPA-2-5 as an exceptional agent for the treatment of sensory discomfort and inflammation arising from non-keratinizng stratified epithelium (NKSE), including mucous membranes, for example, of the upper aerodigestive tract, for example, the oropharyngeal (including, e.g., the pharynx) and upper esophageal surfaces, and anogenital surfaces.

As described herein, DAPA-2-5 is selective and ideal for evoking localized “dynamic cool” in the oropharynx without discomfort. This “dynamic cool” is the desired sensory quality for oropharyngeal/esophageal discomfort [giving a refreshing sensation of cool/cold]. Furthermore, it has good activity in the chili-pepper test. It has anti-inflammatory activity on heat-evoked edema, blocks the effects of acid-stimulated swallowing, and antagonizes the activity of capsaicin on the isolated vagus nerve. These attributes makes DAPA-2-5 an ideal active ingredient to reduce sensory discomfort and inflammation arising from non-keratinizng stratified epithelium (NKSE), especially the membranes of the oropharynx and esophagus.

DAPA-2-5 has selective sensory effects and a localized distribution of this sensation. In an animal model, it was selectively potent in inhibiting the irritant effects of 0.1 N hydrochloric acid on the pharyngeal membranes, a NKSE surface. It has minimal irritancy when it was delivered onto the oral cavity of human volunteers and exerted the desired anti-nociceptive effect. When superfused onto the vagus nerve in vitro, it blocked the depolarization response to capsaicin, a well-known sensory irritant. Its receptive element in the vagus was further characterized as TRPM8, an ion channel receptor.

Furthermore, DAPA-2-5, unlike related analogs, it did not produce stinging, adverse tastes, or “icy cold” or cold discomfort, even when the dose was increased to 8 mg per tablet. The activity of DAPA-2-5 remained localized to the throat and upper esophagus, and there was no systemic cooling. Individuals with throat discomfort preferred DAPA-2-5 because of the immediate onset and the dynamic cool sensation. The “icy cold” seen with other DAPA compounds (DAPA-1-6, DAPA-1-7, DAPA-2-6, and DAPA-2-7) was considered to be too cold, even though these compounds were longer-acting on the throat. The activity of other DAPA compounds (DAPA-1-6, DAPA-1-7, DAPA-2-6, and DAPA-2-7) spreads behind the sternum, into the chest, most likely because of activation of sensory elements in the oesophageal lining.

As compared to a wide range of structurally similar compounds, DAPA-2-5 was found to have the desired sensory qualities for anti-nociception without excessive irritancy, to be highly potent, to have a sufficient duration of action to be therapeutically useful, and also to have anti-inflammatory activity in an animal model.

Chemical Synthesis

DAPA compounds were prepared by the following general method: 100 mL (23.7 g, -200 mmol) of sec-butylmagnesium chloride or bromide (isopropylmagnesium chloride or bromide) (obtained from Acros, as a 25% solution in tetrahydrofuran (THF)) was placed under nitrogen in a 500 mL flask (with a stir bar). Diethylphosphite solution in THF (from Aldrich, D99234; 8.25 g, 60.6 mmol in 50 mL) was added drop-wise. After approximately 30 min, the reaction mixture warmed up to boiling. The reaction mixture was stirred for an extra 30 min, followed by a drop-wise addition of the appropriate n-alkyl iodide solution in THF (from TCI; 60 mmol in 20 mL). The reactive mixture was then stirred overnight at room temperature. The reaction mixture was diluted with water, transferred to a separatory funnel, acidified with acetic acid (˜10 mL), and extracted twice with ether. The ether layer was washed with water and evaporated (RotaVap Buchi, bath temperature 40° C.). The light brown oil was distilled under high vacuum. The final products, verified by mass as determined by mass spectrometry, were clear liquids that were colourless or slightly pale yellow.

The following compounds were prepared by this method:

TABLE 3
Chemical prepared and tested.

| Code | Chemical Name | Chemical Structure |
|---|---|---|
| DAPA-1-5 | 1-Di(isopropyl)- phosphinoyl- pentane | 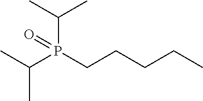 |
| DAPA-1-6 | 1-Di(isopropyl)- phosphinoyl- hexane | 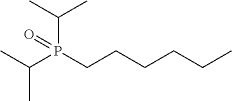 |
| DAPA-1-7 | 1-Di(isopropyl)- phosphinoyl- heptane | 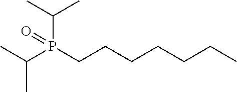 |
| DAPA-1-8 | 1-Di(isopropyl)- phosphinoyl- octane | 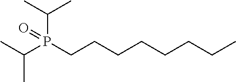 [SMILES: CCCCCCCCP(=O)(C(C)C)C(C)C] |
| DAPA-2-4 | 1-Di(sec-butyl) phosphinoyl- butane | 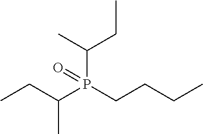 |
| DAPA-2-5 | 1-Di(sec-butyl) phosphinoyl- pentane | 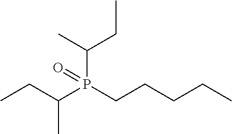 |
| DAPA-2-6 | 1-Di(sec-butyl) phosphinoyl- hexane | 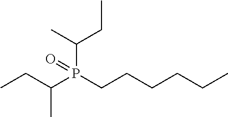 [SMILES: CCCCCCP(=O)(C(C)CC)C(C)CC] |
| DAPA-2-7 | 1-Di(sec-butyl) phosphinoyl- heptane | 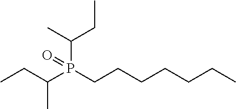 [SMILES: CCCCCCCP(=O)(C(C)CC)C(C)CC] |
| DAPA-2-8 | 1-Di(sec-butyl) phosphinoyl- octane | 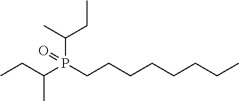 [SMILES: CCCCCCCCP(=O)(C(C)CC)C(C)CC] |
| DAPA-3-1 | 1-di(iso-butyl) phosphinoyl- pentane | 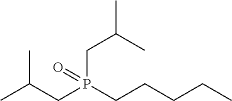 |
| DAPA-3-2 | 1-Di(sec-butyl) phosphinoyl- 3-methyl-butane | 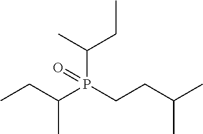 |

Compositions

The discovery also relates to a composition (e.g., a pharmaceutical composition) comprising DAPA-2-5, and a pharmaceutically acceptable carrier, diluent, or excipient.

The discovery also relates to a method of preparing a composition (e.g., a pharmaceutical composition) comprising mixing DAPA-2-5, and a pharmaceutically acceptable carrier, diluent, or excipient.

In one embodiment, the composition comprises DAPA-2-5 at a concentration of 0.005-2.0% wt/vol. In one embodiment, the composition is a liquid composition, and comprises DAPA-2-5 at a concentration of 0.5-20 mg/mL In one embodiment, the composition is a liquid composition, and comprises DAPA-2-5 at a concentration of 1-5 mg/mL. In one embodiment, the composition is a gel composition, and comprises DAPA-2-5 at a concentration of 1-20 mg/mL.

The composition may be provided with suitable packaging and/or in a suitable container. For example, the composition may be in the form of oral dosage unit, for example, a lozenge, jelly cup, edible film strip, or orally disintegrating tablet (ODT) comprising DAPA-2-5.

Similarly, the composition may be provided as a swab, wipe, pad, or towellette (e.g., suitably sealed in a wrap) carrying DAPA-2-5 or a composition comprising DAPA-2-5. Similarly, the composition may be provided as a patch, e.g., a controlled-release patch, e.g., suitable for application to the skin.

Similarly, the composition may be provided as an aerosolized spray delivered from a pressurized container. Similarly, the composition may be provided in a manually-activated sprayer (e.g., with a suitable small orifice) linked to a reservoir containing DAPA-2-5 or a composition comprising DAPA-2-5, for example, capable of delivering a unit volume (e.g., of 0.05 to 0.15 mL), for example, to the skin or a mucous membrane surface.

Use in Methods of Therapy

One aspect of the present discovery pertains to DAPA-2-5 for use in a method of treatment (e.g., selective treatment) of certain disorders (e.g., a diseases), as described herein.

Use in the Manufacture of Medicaments

Another aspect of the present discovery pertains to use of DAPA-2-5 in the manufacture of a medicament for treatment (e.g., selective treatment), for example, treatment (e.g., selective treatment) of certain disorders (e.g., a diseases), as described herein. In one embodiment, the medicament comprises DAPA-2-5. In one embodiment, the medicament comprises DAPA-2-5 formulated as an ODT with a mineral excipient.

Methods of Treatment

Another aspect of the present discovery pertains to a method of treatment (e.g., selective treatment) of certain disorders (e.g., diseases), as described herein, comprising administering to a patient in need of treatment a therapeutically effective amount of DAPA-2-5, preferably in the form of a pharmaceutical composition.

Disorders Treated

In one embodiment (e.g., of use in methods of therapy, of use in the manufacture of medicaments, of methods of treatment), the treatment is treatment (e.g., selective treatment) of: sensory discomfort from non-keratinizng stratified epithelial (NKSE) tissue; upper aerodigestive tract discomfort; oropharyngeal discomfort; esophageal discomfort; throat irritation; cough; heartburn; chest pain; anogenital discomfort; or inflammation of non-keratinizng stratified epithelial (NKSE) tissue.

Disorders Treated—Sensory Discomfort from NKSE Tissue

In one embodiment (e.g., of use in methods of therapy, of use in the manufacture of medicaments, of methods of treatment), the treatment is treatment of (e.g., selective treatment of) sensory discomfort from non-keratinizng stratified epithelial (NKSE) tissue.

The term “sensory discomfort”, as used herein, relates to irritation, pain, itch, or other form of dysesthesias from non-keratinizng stratified epithelial (NKSE) tissue. The term implies activation of nociceptors located on sensory nerve endings in NKSE and bodily tissue and the antagonism of sensations created by the activation of nociceptors. Nociceptors are stimulated, for example, by high or low temperatures, mechanical pressure, chemicals (e.g., capsaicin, acidity, etc.), injury, and inflammatory mediators. A DAPA compound, such as DAPA-2-5, that decreases sensory discomfort, can be termed an anti-nociceptive agent.

The term “dysesthesias” as used herein relates to abnormal sensation, and includes, in addition to irritation, itch, and pain, sensations such as burning, dryness, wetness, pins-and-needles, and feeling the presence of a foreign body.

In one embodiment, the NKSE tissue is located on:
an upper aerodigestive tract surface;
an oral cavity surface;
a respiratory tissue surface;
a nasal membrane surface;
an oropharyngeal surface (including, e.g., a pharyngeal surface);
an esophageal surface; or
an anogenital surface.

In the embodiments, the NKSE tissue is located on an upper aerodigestive tract surface, is located on an oral cavity surface, is located on a lining of the oral cavity; or an internal portion of the lips, on a respiratory tissue surface, is located on a respiratory epithelial surface, is located on a nasal membrane surface, is located on a lumenal lining of a nasal membrane.

In one of the embodiments, the NKSE tissue is located on an oropharyngeal surface, or a pharyngeal surface. In one of the embodiments, the sensory discomfort from NKSE tissue is caused by dysphagia, by reflux of stomach contents (e.g., laryngopharyngeal reflux), by hiccups, by pharyngitis, by tonsillitis, by mucositis, by an allergy, by cough, or by hypersensitivity of the pharyngeal surface to an irritant.

In one of the embodiments, the NKSE tissue is located on an esophageal surface and the sensory discomfort located on an esophageal surface is caused by reflux of stomach contents (e.g., gastroesophageal reflux).

Disorders Treated—Localised Discomfort

In one embodiment (e.g., of use in methods of therapy, of use in the manufacture of medicaments, of methods of treatment), the treatment is treatment of (e.g., selective treatment of) upper aerodigestive tract discomfort.

In one embodiment, the upper aerodigestive tract discomfort is caused by inflammatory exudates in the airways or the pharynx (e.g., associated with asthma, an obstructive pulmonary disorder, etc.).

In one embodiment, the upper aerodigestive tract discomfort is associated with laboured breathing, dyspnea, snoring, or sleep apnea.

In one embodiment, the treatment is treatment of (e.g., selective treatment of) oropharyngeal discomfort.

In one embodiment, the oropharyngeal discomfort is associated with reflux of stomach contents.

In one embodiment, the oropharyngeal discomfort is associated with laryngopharyngeal reflux.

In one embodiment, the treatment is treatment of (e.g., selective treatment of) esophageal discomfort.

In one embodiment, the esophageal discomfort is associated with reflux of stomach contents.

In one embodiment, the esophageal discomfort is associated with gastroesophageal reflux.

In one embodiment, the treatment is treatment of (e.g., selective treatment of) throat irritation.

In one embodiment, the treatment is treatment of (e.g., selective treatment of) cough.

In one embodiment, the treatment is treatment of (e.g., selective treatment of) heartburn.

In one embodiment, the treatment is treatment of (e.g., selective treatment of) chest pain.

In one embodiment, the treatment is treatment of (e.g., selective treatment of) anogenital discomfort.

Disorders Treated—Inflammation of NKSE Tissue

In one embodiment (e.g., of use in methods of therapy, of use in the manufacture of medicaments, of methods of treatment), the treatment is treatment of (e.g., selective treatment of) inflammation of non-keratinizng stratified epithelial (NKSE) tissue.

Treatment

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.”

The term “selective” in pharmacological terminology pertains to a molecule that, among a group of structurally related congeners, exhibits qualitative or quantitative properties that distinguishes it from the other analogs.

The term “selective treatment”, as used herein in the context of treating a disorder with a heat abstraction sensation of “dynamic cool”, pertains to treatment (e.g., suppression) of sensory discomfort from non-keratinizng stratified epithelium (NKSE) without problems of stinging, irritancy, or other adverse effects [e.g. unpleasant tastes in the oral cavity].

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents.

One aspect of the present discovery pertains to DAPA-2-5 in combination with one or more (e.g., 1, 2, 3, 4, etc.) additional therapeutic agents. The particular combination would be at the discretion of the physician who would select dosages using his common general knowledge and dosing regimens known to a skilled practitioner.

Examples of additional therapeutic agents include: anti-inflammatory steroidal agents; anti-inflammatory analgesic agents; antihistamines; sympathomimetic amine vasoconstrictors; local anesthetics; antibiotics; anti-acne agents; topical retinoids;

drugs for cough; drugs for mucous secretion; drugs for dysphagia, including spices; drugs for genital warts; drugs for wrinkles; drugs for ageing skin; anti-hemorrhoidal agents; drugs for vulvar itch; skin moisturizers; and agents for treating keratolysis.

Examples of steroidal anti-inflammatory agents include: hydrocortisone, clobetasol, clobetasol propionate, halobetasol, prednisolone, dexamethasone, triamcinolone acetonide, fluocinolone acetonide, fluocinonide, hydrocortisone acetate, prednisolone acetate, methylprednisolone, dexamethasone acetate, betamethasone, betamethasone valerate, flumetasone, fluticasone, fluorometholone, and beclomethasone dipropionate.

Examples of anti-inflammatory analgesic agents include: methyl salicylate, monoglycol salicylate, aspirin, indomethacin, diclofenac, ibuprofen, ketoprofen, naproxen, pranoprofen, fenoprofen, sulindac, fenclofenac, clidanac, flurbiprofen, fentiazac, bufexamac, piroxicam, and pentazocine.

Examples of antihistamines include: diphenhydramine hydrochloride, diphenhydramine salicylate, diphenhydramine, chlorpheniramine maleate, and promethazine hydrochloride.

Examples of sympathomimetic amine vasoconstrictors include: phenylephrine hydrochloride, oxymetazoline, naphazoline, and other imidazoline receptor agonists used for nasal decongestant activity.

Examples of local anesthetics include: dibucaine hydrochloride, dibucaine, lidocaine hydrochloride, lidocaine, benzocaine, pramoxine hydrochloride, tetracaine, tetracaine hydrochloride, oxyprocaine hydrochloride, mepivacaine, and piperocaine hydrochloride.

Examples of drugs for cough and drugs for mucous secretion include: dextromethorphan, dextromethorphan hydrobromide, codeine, dichloropheniramine, guaifenesin, and phenol.

Examples of skin moisturizers include the three categories of humectants, emollients and preservatives. Humectants, such as urea, glycerin, and alpha hydroxy acids, help absorb moisture from the air and hold it in the skin. Emollients, such as lanolin, mineral oil, and petrolatum, help fill in spaces between skin cells, lubricating and smoothing the skin. Preservatives help prevent bacteria growth in moisturizers. Other ingredients that moisturizers may contain include vitamins, minerals, plant extracts, and fragrances.

Examples of antibiotics include: neomycin, erythromycin, and the anti-viral agent docosanol (Abreva®).

Examples of topical anti-acne agents include: benzoyl peroxide, resorcinol, resorcinol monoacetate, phenol, and salicylic acid.

Examples of topical retinoids include: adapalene and isotretinoin (Retin-A, Differen, and Tazorac). Examples of keratolytics include: alpha-hydroxy acids, glycolic acid, and salicylic acid.

Kits

One aspect of the discovery pertains to a kit comprising (a) DAPA-2-5, or a composition comprising DAPA-2-5, e.g., preferably provided in a suitable container and/or with suitable packaging; and (b) instructions for use, e.g., written instructions on how to administer the compound or composition.

The written instructions may also include a list of indications for which the active ingredient is a suitable treatment.

The written instructions (e.g., pamphlet or package label) may include the dosage and administration instructions, details of the formulation's composition, the clinical pharmacology, drug resistance, pharmacokinetics, absorption, bioavailability, and contraindications.

Methods of Diagnosis

DAPA-2-5 may also be used in differential diagnosis of chest pain. More specifically, DAPA-2-5 may be used to differentiate pain of cardiac versus pain of non-cardiac origin. A simple diagnostic tool of this type is not known to the prior art. A DAPA compound, such as DAPA-2-5 or DAPA-2-7, administered orally, e.g., as a lozenge or orally disintegrating tablet (ODT), can be used to provide differential diagnosis of chest pain, e.g., for differentiating non-cardiac chest pain (NCCP) from cardiac pain.

Routes of Administration

The DAPA-2-5 or pharmaceutical composition comprising DAPA-2-5 may suitably be administered to a subject topically, for example, as described herein.

The term “topical application”, as used herein, refers to delivery onto surfaces of the body in contact with air, which includes the skin, the anogenital surfaces, the transitional epithelial surfaces of the orbit, the lips, the nose, and the anus, and the aerodigestive tract (nasal membranes, pharyngeal and esophageal surfaces), lower respiratory tracts, and the lumen of the gastrointestinal tract.

In one embodiment (e.g., of use in methods of therapy, of use in the manufacture of medicaments, of methods of treatment), the treatment is treatment by topical administration.

In one embodiment, the treatment is treatment by topical administration to non-keratinizng stratified epithelial (NKSE) tissue, as described herein.

For example, in one embodiment, the NKSE tissue is located on:
an upper aerodigestive tract surface;
an oral cavity surface;
a respiratory tissue surface;
a nasal membrane surface;
an oropharyngeal surface (including, e.g., a pharyngeal surface);
an esophageal surface; or
an anogenital surface.

For example, in one embodiment, the NKSE tissue is located on:
an oropharyngeal surface (including, e.g., a pharyngeal surface);
an esophageal surface; or
an anogenital surface.

The Subiect/Patient

The subject/patient may a mammal, for example, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

In one preferred embodiment, the subject/patient is a human.

Formulations

While it is possible for DAPA-2-5 to be administered alone, it is preferable to present it as a pharmaceutical formulation (e.g., composition, preparation, medicament) comprising DAPA-2-5 together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents.

Thus, the present discovery further provides pharmaceutical compositions, as described above, and methods of making pharmaceutical compositions, as described above. If formulated as discrete units (e.g., wipe, pads, towellettes, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Dosage

It will be appreciated by one of skill in the art that appropriate dosages of DAPA-2-5, and compositions comprising DAPA-2-5, can vary from patient to patient. Determining the optimal dosage will generally involve the balancing of the level of therapeutic benefit against any risk or deleterious side effects. The selected dosage level will depend on a variety of factors including, but not limited to, the activity of DAPA-2-5, the route of administration, the time of administration, the duration of the treatment, other drugs, compounds, and/or materials used in combination, the severity of the disorder, and the species, sex, age, weight, condition, general health, and prior medical history of the patient. The amount of DAPA-2-5 and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, preferably on an “as-need” or pro re nata basis throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the patient, treating physician, veterinarian, or clinician.

Upper Aerodigestive Tract

The oral cavity contains specialized structures such as teeth, gums, tongue, and salivary glands that are designed to masticate, taste, lubricate, and propel the food bolus into the pharynx. This is one of the most complicated muscular reflex activity in the body and requires the coordination at least 6 cranial nerves and 25 muscle groups. Heat sensation is not a high ranking protective reflex in the oral cavity as the mouth can tolerate hot liquids which are painful when put on the skin. Cooling liquids, on the other hand, is important in the regulation of thirst and detection of cooling has survival value. Eccles et al. [ Cold pleasure. Why we like ice drinks, ice-lollies and ice cream. Appetite, 71, 357-60, 2013] .recently reviewed this concept on the relationships of cooling liquids, ice creams, positive reinforcement, and the suppression of thirst. Sensory nerves closely monitor temperatures at the junction of the oral cavity and pharynx. When the external ambient temperature is high, drinking cooling liquids is instantly pleasurable and relieves thirst, dryness, and discomfort.

The pharynx is a cone-shaped passageway leading from the nasal and oral cavities to the larynx and esophagus. The pharynx is part of the throat, an inexact term describing the region of the body around the neck and voice-box. The pharynx is divided into three regions: naso-, oro- and laryngo-. The nasopharynx, also called the rhinopharynx, lies behind the choanae of the nasal cavity and above the level of the soft palate. The oropharynx reaches from the soft palate (velopharynx) to the level of the hyoid bone. The laryngopharynx reaches from the hyoid bone to the lower border of the cricoid cartilage. The pharyngeal surfaces are lined with non-keratinizng stratified epithelium (NKSE).

The oropharynx may be further divided into an upper and lower region, the mid-point being what is called the lower retropalatal oropharynx (LRO) as shown, for exampe, in the magnetic resonance imaging studies of Daniel et al. [“Pharyngeal dimensions in men and women”, Clinics (SaoPaulo) 62, 5-10, 2007]. The pharynx is a trapezoid inverted funnel-shaped tube and the LRO is the region with smallest cross-section, an area of about 1 cm², which is equivalent to 20% of US quarter coin of 25% of a Euro coin. The pharyngeal surfaces at the base of the tongue and the pharyngeal wall around the LRO, an area of about 3 to 5 cm², are one part of the desired target for drug delivery for the methods described herein, the second part being the upper esophageal surface.

The lumen of the oropharynx is a conduit for food, liquid, and air, and is part of both the digestive and respiratory systems and is also called part of the aerodigestive tract (an anatomical term defined by the International Health Terminology Standard Development Organisation). The traffic that passes through the oropharynx every day is astounding. On an average day, an adult breathes 12,000 L of air, drinks 2 L of fluids, secretes 1 L of saliva, and eats 2 kg of food. These activities are constant, with about 15 breaths and 1 swallowing movement per min during the waking hours.

For the organism to survive, the traffic flow must be co-ordinated so that food and liquids go down the esophagus and not into the airways, and air gets directed into the airways. The efficiency of this system is visible and self-evident, for example, when a large pizza is consumed with a soft drink. The transit of mass from mouth to stomach is accomplished with a minimum of fuss.

The brain is the traffic co-ordinator for the pharynx and the effectors are striated and smooth muscles. At least 6 cranial nerves and 25 muscles participate in swallowing. For solids, the food is masticated, mixed and lubricated with secreted saliva, and then the bolus is then rapidly pushed down to the esophagus. The oropharyngeal phase of swallowing occurs in the blink of an eye, in millisec, as the bolus transits down the pharynx at about 35 cm/sec. The sensory signals that govern this process in the mouth and rostral tongue come from afferent signals of branches of the trigeminal (5^{th}) nerve and the hypoglossal (8th) nerve. The afferent signals from the oropharynx and posterior surface of the tongue come mainly via glossopharyngeal nerve (9^{th}). Signals from the laryngopharynx are via the vagus nerve (10^{th}). Swallowing and coughing (when things go the wrong way) are reflexes coordinated by the cranial nerves and muscles that are designed to direct the traffic load to their correct destinations.

The neuronal receptive fields of the epithelia (naso-, oro-, laryngo-, upper oesophageal-, and bronchial epithelia) of the upper aerodigestive tract (oral and nasal cavities, pharynx, upper airways and esophagus), are mainly sub-served by the 5^{th}, 9^{th} and 10^{th} cranial nerves. These surfaces are mainly lined by mucosa, i.e., non-keratinizng stratified epithelium (NKSE). These cells have a high turnover rate (on the order of several days) and are sensitive to injury. For example, when there is disorganized traffic of solids or liquids in the pharynx, acid and pepsin, or exudates from the lungs, accumulation in the aerodigestive tract will activate the cranial nerves and cause irritation, itch, pain, and inflammation. The characteristic manifestations of pharyngeal disorders are globus (the feeling of a lump in the throat), difficulties in swallowing (dysphagia), hoarseness, pain, itch, cough, and redness and swelling of the pharyngeal mucosa.

The pharynx has strong, constrictor muscles, arranged as a vice and designed to grab the oropharyngeal contents and push the bolus into the esophagus. The anatomy is like the first baseman glove in baseball. There are two important valves in this system: the epiglottis which closes during swallowing, and the upper oesophageal sphincter (UES, or cricopharyngeus muscle) which relaxes to allow the contents to enter the esophagus, then shuts to prevent reflux. Pharyngeal contraction flushes and empties the lumen of debris, and by creating negative pressure helps suck contents from the nasal cavity and nasopharynx. Well-toned pharyngeal muscles are also important for maintaining patency of the airways, allowing smooth airflow and dysfunction will cause snoring, and sleep apnea.

Examples of upper aerodigestive disorders in which a topical anti-nociceptive agent may have utility are:

Pharyngitis: An inflammation of the pharyngeal lining which is most commonly caused by viral and bacterial agents. A closely related condition is tonsilitis [Bathala, S. and Eccles, R. A review on the mechanism of sore throat in tonsillitis. Journal of Laryngology and Otology, 127: 227-32, 2013]. Chemical pollutants, such as cigarette smoke, can also directly irritate and damage the mucosa. The principal symptoms of pharyngitis and tonsillitis are irritation, itch, and pain or a “sore throat”. Prolonged pharyngeal irritation can also lead to a chronic hypersensitivity syndrome manifested by persistent cough (called chronic cough when it is present for more than 8 weeks). The agents described herein will relieve the discomfort of pharyngitis and potentially reduce inflammation.

Dysphagia (swallowing dysfunction): A common affliction in the elderly, stroke victims, individuals with Parkinson's disease, and individuals with head and neck cancer. Oropharyngeal dysphagia is a term applied to the condition where the bolus of food is not properly transferred from the pharynx to the esophagus. When particles enter the airways, the result is aspiration pneumonia, a major economic burden in the care of such victims. It has been shown that sensory stimulants such as black pepper, capsaicin-like substances (the active ingredients of chili pepper) administered with a nebulizer, and menthol solutions administered by a nasal tube, shortened the latency for a swallowing reflex in the elderly and thus may be employed to reduce the risks of aspiration pneumonia [Ebihara et al., Sensory stimulation to improve swallowing reflex and prevent aspiration pneumonia in elderly dysphagic people”, J. Pharmacol. Sci., 115, 99-104, 2011]. A related condition is called aspiration pneumonitis, when the substances entering the airways come from the esophagus and not the oral cavity.

Post-nasal drip: A condition where there is increased secretions entering the orpharynx from the mucosa of the nasal cavities and nasopharynx. These secretions may contain inflammatory exudates and may arise from infections or allergy of nasal membranes (for example, allergic rhinitis, and rhinosinusitis). The increased secretions cause throat discomfort, pain, itch, cough, and a sense of impaired airflow. An anti-inflammatory or anti-nociceptive agent delivered to the oropharyngeal mucosa will have therapeutic value in this condition by reducing the sense of pharyngeal irritation.

Laryngopharyngeal reflux disease (LPR) and esophageal reflux disease: Conditions where acid and pepsin regurgitate from the stomach into the pharynx. Normally, proper deglutition and a constricted upper oesophageal sphincter (UES), prevent regurgitation, but when this system is impaired, the acid and pepsin enters the pharyngeal surfaces and can even enter the Eustachian tubes and the nasal sinuses. The result is a syndrome of hoarseness, pain, laryngoedema, and persistent throat clearing. Examination of the larynx shows red and swollen mucosae about the voicebox. A sensory agent that decreases surface inflammation is likely to be useful in the treatment for LPR.

Acid reflux disease: A condition similar to LPR [Oustamanolakis et al., Dyspepsia: Organic vs functional. J. Clin. Gastroenterol., 46, 175-190, 2012]. This condition consists of symptoms in the upper abdomen, such as fullness, discomfort, early satiation, bloating, heartburn, belching, nausea, vomiting, or pain. Disorders of the upper digestive tract are further sub-divided into “organic” and “functional dyspepsia”. Organic dyspepsias (OD) are caused, for example, by peptic ulcer, gastroesophageal reflux disease (GERD), Barrett's esophagus, gastric or esophageal cancer, pancreatic or biliary disorders, intolerance to food or drugs, and infections or systemic diseases. Functional dyspepsias (FD), including NERD, are more complex because objective evidence of pathology is not easily identified, but the symptoms are similar to OD. Management of these conditions includes acid-suppressive drugs, antibiotics to eradicate H. pylon, prokinetic agents, fundus-relaxing drugs, antidepressants, and psychological interventions.

Epigastric discomfort in the upper digestive tract: A condition that includes the symptom called “heartburn” and non-cardiac chest pains which are the predominant symptoms of acid reflux disorders. The pain and discomfort of heartburn is primarily of oesophageal origin. Non-cardiac chest pain is pain of esophageal origin and not caused by cardiac dysfunction. Some of the symptoms of these conditions are described as heartburn (a burning feeling in the chest just behind the breastbone that occurs after eating and lasts a few min to several hours). The substernal burning sensations tend to radiate up into the neck, come in waves, and are felt more as burning than as pain. Heartburn may also be described as chest pain and is exaggerated by which is exaggerated by assuming positions which promote gastroesophageal regurgitation, such as bending over or lying on one's back. Heartburn is felt in the midline and not on the lateral sides of the chest. Other sensations include burning on or at the back of the throat with sour, acidic or salty-tasting fluids in the mouth and throat; difficulty in swallowing and feelings of food “sticking” in the middle of the chest or throat. Heartburn and acid reflux diseases may cause chronic cough, sore throat, or chronic hoarseness.

Excess reflux of acidity and digestive enzymes such as pepsin into the esophagus and pharynx give rise to the discomfort seen in GERD, laryngopharyngeal reflux disease (LPR), non-erosive reflux disease (NERD), non-cardiac chest pain (NCCP), and functional dyspepsias.

A provocation test, using 0.1 N HCl perfusion of the esophagus alternating with saline perfusion (Bernstein test), can be used to elicit heartburn in susceptible individuals and to prove esophageal origin of the symptoms, e.g., to determine if chest pain is caused by acid reflux. For this test, a thin tube is passed through one nostril, down the back of the throat, and positioned into the middle of the esophagus. A 0.1 N hydrochloric acid solution and a normal salt solution are alternately infused through the catheter and into the esophagus, for example, at the rate of 8 mL/min for 10 min. The patient is unaware of which solution is being infused. If the perfusion with acid provokes the patient's usual pain and perfusion of the salt solution produces no pain, it is concluded that the patient's pain is related to acid reflux. Using this objective method of assessment, complaints of discomfort to HCl perfusion were noted in 7% of normal subjects, 17% in Barrett's esophagus, 32% in GERD, and 58% in NERD patients. In the Examples, an animal model is used to demonstrate that DAPA-2-5 acts as anti-nociceptive agents against the irritative effects of 0.1 N hydrochloric acid.

Chest Pain and Differential Diagnosis of Chest Pain

Chest pain, accompanied sometimes by palpitations, sweating, shortness of breath, and choking sensations, is a common symptom that provokes a patient to see a physician or to seek admission to an Emergency Department. The physician's first priority on examining the patient is to determine if there are any life-threatening cardiovascular conditions. If warranted, a hospital admission for chest pain can be expensive because of work-up diagnostics such as serum enzyme assays, electrocardiograms, and radiotracer studies on heart function. It has been noted that the median cost of a hospital admission for a patient with chest pain was US$7340 [Coley et al., Economic burden of not recognizing panic disorder in the emergency department. J. Emergency Medicine_{—}36: 3-7. 2009]. Each year, approximately 6.4 million Americans visit the Emergency Department with complaints of chest pain and related symptoms, but only a small percentage exhibit an underlying cardiovascular etiology; the others have non-cardiac chest pain (NCCP). Chest pain is the second most common reason for an Emergency Department visit, the first reason being stomach and abdominal pain [see, e.g., Table 8 in Pitts et al., National Hospital Ambulatory Medical Care Survey: 2006 emergency department summary”, National Health Statistics Reports, Vol. 7, pp. 1-38, 2006].

There are multiple causes of NCCP, including pectoral muscle strain, pulmonary disorders, indigestion, panic disorders, and, most frequently, esophageal dysfunction such as GERD [Amsterdam et al., Testing of low-risk patients presenting to the emergency department with chest pain: a scientific statement from the American Heart Association. Circulation, 122: 1756-1776, 2010]. Standard proton pump inhibitor drugs such as esomeprazole has very limited efficacy in suppressing unexplained chest pain and the onset of drug effect requires at least several days [Flook et al., Acid-suppressive therapy with esomeprazole for relief of unexplained chest pain in primary care: a randomized, double-blind, placebo-controlled trial, Amer. J. Gastroenterol., 108: 56-64, 2013].

A simple test, to distinguish NCCP from cardiac pain, may aid in the differential diagnosis of chest pains, permit triage of patients, and improve allocation of resources to reduce the costs of care. In a case study described herein, an elderly subject played a vigorous round of golf, then ate and drank too much, and started experiencing pain in the retrosternal and left pectoral region of the chest. He took Alka-Seltzer and an antacid and tried lying down, but the pain did not go away. Before making the decision to call for Emergency Services, he swallowed three tablets, each containing 1.5 mg of DAPA-2-5, and, surprisingly, his chest discomfort was relieved within 5 min. This dramatic effect is, however, consistent with the pharmacology of DAPA-2-5, namely, to exert an anti-nociceptive effect on cranial nerve endings of the upper digestive tract.

It is proposed here that an active ingredient such as DAPA-2-5, delivered onto the surface of the upper digestive tract, may be useful for the relief of chest pain and aid in the differential diagnosis of chest pains. Agents that counteract the effects of acid on the pharynx and esophageal such as DAPA-1-7, DAPA-1-8, DAPA-2-6, and DAPA-2-7 may also be used for this purpose because they are anti-nociceptive on the nerve endings of the NKSE and will antagonise NCCP. Thus, such agents may be used for the short-term management and differential diagnosis of chest pain.

Diseases of the Airways

Diseases of the airways, such as asthma, chronic obstructive pulmonary disease, and bronchitis, are associated with inflammation of airway mucosa and increased production of exudates. Exudates are normally removed by expectoration or swallowing. At night and during sleep, the pharyngeal muscles relax and clearance is inhibited, so exudates may accumulate in the oropharynx, and cause choking and gagging. A sensory agent that counteracts discomfort in the oropharynx and airways will be useful for such airway diseases.

In the studies described herein, it is shown that DAPA-2-5 superfused onto the isolated vagus nerve directly inhibits capsaicin-induced depolarization. Thus, it is shown that an agent delivered onto the afferents of the 9^{th} and 10^{th} nerves has the potential to counteract oropharyngeal and upper esophageal discomfort. Surprisingly, DAPA-2-5 also manifested anti-inflammatory activity in a model of heat injury and thus may have value in the treatment of inflammation of the NKSE.

There have been a limited number of attempts to treat the upper aerodigestive tract with sensory agents. It has been proposed to use sensory agents such as black pepper, lavender, capsaicin, capsids, and menthol to treat the dysphagia problems of the elderly [Ebihara et al., 2011]. These agents were applied as aerosolized liquid suspensions, or as a liquid delivered via a nasal tube to the pharynx. The exact sensory event for enhancement of clearance reflexes was not defined. Potent menthol and peppermint oil confectionery, such as Altoids®, are also sensory stimulants in the oral and nasal cavities. Menthol lozenges, weighing about 2.7 to 3.4 g each, and containing 5, 7, or up to a maximum of 10 mg of menthol in a sugar-dye matrix, are also sometimes used as oral stimulants, but have limited efficacy because of harsh taste. Certain N-alkyl-carbonyl-amino acid esters have been described for use in the treatment of throat discomfort and airway irritation [Wei, US 20110082204 A1, 2011 and US 084226463, Apr. 23, 2013]. Compounds with desirable properties are (R)-2-[((1R,2S,5R)-2-isopropyl-5-methyl-cyclohexane-carbonyl)-amino]-propionic acid ethyl ester and [((1R,2S,5R)-2-isopropyl-5-methyl-cyclohexanecarbonyl)]-amino-acetic acid isopropyl ester].

In the context of the present discovery, the goals were to:
(a) Define an active compound with a precise anti-nociceptive sensation in the membranes of the upper aerodigestive tract that will counteract discomfort (irritation, itch, and pain). This sensation will not, of itself, produce discomfort but instead generate a sensation called “dynamic cool”, similar to when ice cream is swallowed. For each tested compound, the sensation to avoid is a condition referred to as “cold discomfort”.
(b) Develop a topical formulation for localized delivery of the active compound onto targets of the nerve endings of one or more of the 5^{th}, 9^{th}, and 10^{th} cranial nerves.
(c) Define a drug action with rapid onset (less than 10 sec) and long duration (effective for at least several hours), with a dosage schedule that can be based on an “as needed” basis (pro re nata or p.r.n.), and thus allowing the patient to regain control of the sensory discomfort. Ideally, the active compound is potent, with a unit dose of less than 5 mg per administration.
(d) Define an active compound with the additional benefit of an anti-inflammatory effect on epithelial tissues.

These objectives are met with formulations containing DAPA-2-5, e.g., 1 to 5 mg of DAPA-2-5.

Targeted Topical Delivery onto a Specific Location

To create a drug for topical delivery to the pharyngeal and esophageal surfaces requires careful understanding of the target tissues and the dynamics of the tissue environment. If the cooling ingredient is just for toothpaste and chewing gum then dissolving the ingredient in saliva and delivering it to the nerve endings of the lingual branch of the 5th nerve [trigeminal, which innervates the tongue and buccal mucosa] is sufficient. By contrast, the neuronal receptive fields of the pharynx, larynx, and esophagus are linked to the afferents of the 9^{th} [glossopharyngeal], 10^{th} [vagus], and spinal afferents, and have different anatomy.

The oropharynx is the arch-shaped structure at the base of the tongue, with the uvulva [or grape] hanging in the middle. The base of the arches, called the anterior pillars of fauces, is especially sensitive to cold sensations. If a cold metal probe is placed at this site in human subjects, cooling sensations and rapid swallowing movements are elicited [Kaatzke-McDonald, E. et al. The Effects of Cold, Touch, and Chemical Stimulation of the Anterior Faucial Pillar on Human Swallowing. Dysphagia 11:198-206,1996]. The pharynx and laryngx surfaces are densely innervated by nerve endings of 9^{th} and 10^{th} cranial nerves [Mu and Sanders. Sensory nerve supply of the human oro- and laryngopharynx: a preliminary study. The Anatomical Record, 258, 406-20, 2000]. TRPM8 immunoreactive fibers are found in the lingual nerve of the tongue [Abe, J. et al. TRPM8 protein localization in trigeminal ganglion and taste papillae. Brain Research. Molecular Brain Research, 136: 91-8, 2005], but are especially abundant at the border of the oropharynx, and in the larynx, but not in the epiglottis [Sato, T. et al. The distribution of transient receptor potential melastatin-8 in the rat soft palate, epiglottis, and pharynx. Cellular and Molecular Neurobiology, 33:161-5, 2013]. The desired drug targets are the receptive fields of 9^{th} and 10^{th} nerve.

The throat is a term describing the region of the body around the voice-box. Internally, the relevant structure is the pharynx which is divided into three sections: naso, oro and laryngo. The nasopharynx, also called the rhinopharynx, lies behind the nose and above the level of the soft palate. The oropharynx reaches from the soft palate (velopharynx) to the level of the hyoid bone. The laryngopharynx is in the space behind the larynx and reaches from the hyoid bone to the lower border of the cricoid cartilage. The oro- and laryngo- pharynx is a continuous funnel-shaped inverted trapezoid tube [Daniel et al., 2007] and the total surface area is about 10 to 15 cm².

The favored targets for drug delivery are rostral surfaces of the oropharynx, on the soft palate, the pillars of fauces, and the lateral oro-pharyngeal walls. A second target site is the lumen of the upper esophagus. To reach the upper esophageal linings, the formulation must get past the upper esophageal sphincter without a long residence in the laryngopharynx.

The afferent signals to the brainstem from the posterior surface of the tongue, the oropharynx, and the laryngopharynx are primarily from the 9^{th} (glossopharyngeal) and 10^{th} (vagus) cranial nerves, with a few fibres from the 7^{th} (facial) cranial nerve. The afferent signals from the receptive fields coordinate the clearance reflexes that empty the pharynx and protect the airways against entry of liquids and solids. For the upper esophagus, the innervation is from the vagus and spinal afferents. The targets for drug delivery are primarily the receptive fields of the 9^{th} and 10^{th} cranial nerves, and, to a lesser extent, the 7th nerve and the spinal afferents of the upper esophagus.

The oropharyngeal phase of swallowing occurs in the blink of an eye, in millisec, as the bolus moves from mouth to esophagus. The transit time, as measured by laser Doppler ultrasound or X-ray videofluorography is about 35 cm/sec [Sonomura et al., Numerical simulation of the swallowing of liquid bolus. J. Texture Studies 42: 203-211, 2011]. It is therefore difficult to deliver (coat onto) and retain a sensory agent on the surface of the oro-laryngopharynx. The active ingredient cannot be delivered as solid particles, as that would cause irritation and elicit coughing, so delivery in a liquid, spray, or solute in saliva is the ideal method.

The methods of topical drug delivery are further described in a separate section of this application.

Onset, Duration of Action, and Schedule of Delivery

As contemplated here, the delivered agent for treatment should have a sensory effect with rapid onset of action, for example, less than 10 sec and no more than 2 min.

The effects should be effective for at least one hour and preferably longer, otherwise the patient would have to repeatedly apply the drug to obtain relief. Preferably, there should be a “wow effect” of the active ingredient to stimulate sensory events. The patient should be able to identify this “wow effect” and use the ODT, liquid, or spray on an “as needed” (p.r.n.) basis. In the context of the present discovery, the “wow effect” is called “dynamic cool”. With a fast onset of action, the patient should be able to be relieved of oropharyngeal and upper esophageal discomfort, and this relief will further reduce psychogenic factors (e.g., anxiety) associated with throat discomfort. These goals are achieved by DAPA-2-5.

Selection of Active Ingredient: Molecular Target

There is a general view that the ion channel receptor called TRPM8 receptor is the principal physiological element that responds to sensory/cooling agents such as menthol and icilin [McKemy et al., Identification of a cold receptor reveals a general role for Trp channels in thermosensation, Nature, 416, 52-58, 2002].

TRPM8 is a protein with 1104-amino acid residues and has six transmembrane domains. Activation of this receptor by decreasing ambient temperature results in non-specific cation entry into the cell. Depolarization of sensory neurons may then transmit signals to the brain primarily via A6 (and some C) fibres. While this concept for the role of TRPM8 in sensory physiology may be valid for physical changes in temperature, the interpretation of the sensory effects of chemical agents such as menthol and icilin are more complex. Menthol not only stimulates TRPM8 in vitro, but also TRPV3, a receptor associated with warmth [Macpherson et al., More than cool: promiscuous relationships of menthol and other sensory compounds. Mol Cell Neurosci 2006; 32:335-343, 2006]. Menthol also inhibits TRPA1. Icilin stimulates not only TRPM8, but also TRPA1, and icilin inhibits TRPV3 [Sherkheli et al., Supercooling agent icilin blocks a warmth-sensing ion channel TrpV3. Scientific World Journal 2012; 982725, 2012] and glycinergic transmission [Cho et al. TrpA1-like channels enhance glycinergic transmission in medullary dorsal horn neurons. J Neurochem 122:691-701.2012]. Thus, menthol and icilin are “promiscuous” drugs and their specific sensory effects may not be associated with any one particular receptor protein.

The correlation between a chemical's potency at the TRPM8 receptor (measured by the EC₅₀ μM) and potency to evoke sensory events in the oropharynx is complex. The Inventor studied 21 compounds (including the 11 DAPA compounds described herein, menthol, icilin, 7 p-menthane carboxamide amino acid esters, and 1 p-menthane carboxylester), covering a 100-fold range of TRPM8 potency, each of which exhibited full efficacy at the TRPM8 receptor, and evaluated their sensory effects. Surprisingly, a number of side-effects were observed with some of the compounds. For example, menthol, which ranked 16^{th} in TRPM8 potency among the 21 compounds tested, produced chest discomfort at a dose of 2 mg in an ODT. By contrast, icilin, which ranked 4^{th} in TRPM8 potency among the 21 compounds tested, did not produce cooling in the chest or the desired sensations on the throat. Three p-menthane carboxamide amino acid esters, which ranked 1^{st}, 5^{th}, and 13^{th} in TRPM8 potency among the 21 compounds tested produced comfortable cooling in throat. However, only one of them had the desired “refreshing/dynamic cooling” on the oropharynx. Among the DAPA compounds, the relationships of TRPM8 receptor potency to sensory events were not easily categorized. Surprisingly, DAPA-2-5, which has all of the desirable qualities for an active ingredient, ranked 12^{th} in TRPM8 potency among the 21 compounds tested.

As shown in Study 4, the EC₅₀ [median effective dose] of a candidate for activating TRPM8 has little predictive value in identifying a candidate for treatment of sensory discomfort in the upper digestive tract. This is not surprising and to over-interpret the EC₅₀ value is naïve. The 95% Confidence Limits of many EC₅₀ values overlap and only analogs with at least 5× difference in potency are clearly distinguishable in sensory effects from each other. The EC₅₀ values do not give information on the quality of the heat abstraction sensation, the duration of action, or the likelihood of unpleasant taste. Thus, identification of selective agents requires other bioassays that are more able to directly address these questions.

Recently, it has been suggested that there are distinct groups of TRPM8 expressing neurons that separately mediate the effects of innocuous cool, anti-nociceptive activity, and cold pain [Knowlton et al., A sensory-labeled line for cold: TRPM8-expressing sensory neurons define the cellular basis for cold, cold pain, and cooling-mediated analgesia. J Neurosci 33, 2837-2848. 2013]. The sensory effect of a given TRPM8 agonist would then be a balance of the stimulant actions on each subset of neurons. DAPA-2-5 may be an eclectic agonist, selectively producing innocuous cool and anti-nociception, without causing irritation/pain or adverse tastes.

Exact Desired Sensation in Throat to Treat Discomfort

When it became clear that TRPM8 receptor potency screening could not be used as the primary method for selection of an active ingredient, it was necessary to develop alternative methods of bioassays. A precise definition of the desired sensation in the throat was necessary to set the stage for further testing.

When a test compound is applied to non-keratinizng bodily surfaces (e.g., oropharyngeal surface), it is possible to characterize the resulting sensations. The quality of the sensations produced by individual compounds favours certain characteristics that are distinct. The quality of the sensations evoked, their descriptors, and their proposed mechanism of action, are summarised in the following table. For any compound, there may be some overlap in activity, but usually one compound occupies only one or two categories of sensations.

TABLE 4
Description of sensations of heat abstraction and machenisms.

| | | Mechanisms on |
|---|---|---|
| Type of Sensation | Descriptor | Sensory Neurons |
| Inactive | No effect | — |
| Cool, steady and pleasant | Cool | Balanced stimulation of |
| | | static and dynamic |
| Cold, constant, but limited by | Cold | Higher stimulation of static |
| desensitization | | |
| Dynamic cooling, robust | Dynamic | Higher stimulation of |
| cool/cold, strong refreshing | cool | dynamic |
| Stinging cold, sometimes with | Icy cold | Stimulation of dynamic and |
| irritation | | static, and also nociceptive |
| | | sites |

Some of the DAPA compounds studied evoked sensations of intense cold in the oral cavity. The sensations are akin to rapid drinking of cold water mixed and equilibrated with ice chips. The intense cold is further accentuated if the drink is acidified, for example, with lemonade. The sensations of dull and intense cold on the surface of the oropharynx can be described as painful, uncomfortable, and aversive. The term “icy cold” is used to describe these adverse intense cold sensations.

A second type of cold discomfort noted, for example, with DAPA-1-6, DAPA-2-6, and DAPA-2-7, was sensations of cold in the chest. The feeling of cold was behind the sternum and in the upper thorax. Most likely, the compound rapidly distributed and activated cold sensations in the oesophageal lining. These sensations were considered unpleasant by some subjects, but may have utility in the treatment of heartburn and chest pain.

At higher oral doses (e.g., 5 mg or more) of DAPA-2-7, it was also noted that there were sensations of cold on other body surfaces. The facial skin and the surface of the eyeball felt coolness and cold. The surface skin of the scapula and the ankles also felt coolness and cold, especially if there was a draft (increased airflow) in the room. The hands felt cold, as if the blood vessels were constricted. These sensations could have resulted from the systemic absorption of the DAPA-2-7 into the bloodstream. Alternatively, it is possible that strong coolness at one site may make the brain “generalize” the sensation, and attribute coldness to other parts of the body. These systemic sensations of cold, if not expected by the test subject, can be alarming and viewed as unpleasant.

Together, these three types of sensations—“icy cold”, coldness in the chest, and systemic coldness—is termed “cold discomfort”. Cold discomfort limits selection of the active ingredient for an agent designed for localized action on the oropharynx/upper esophagus. The ideal agent must have a circumscribed site of action, and the intensity of the sensation should not cause “icy cold”, coldness in the chest, or systemic chills.

The oral cavity, throat, and upper esophagus can feel coolness, chill, and cold. This is a fact of human experience. When ice cream is placed in the mouth, there are pleasant cooling and sweet sensations on the tongue and on the walls of the mouth. When the ice cream is swallowed there is a very brief (one or two sec at most) robust refreshing sensation on the back of the mouth. This sensation in the upper throat can be replicated by repetitive swallowing or sipping of ice cream, or the equivalent sipping of a “milk shake” or “smoothie”. This is the desired sensation for treating oropharyngeal/esophageal discomfort. This sensation is described herein as “dynamic cool” and is distinct from cool, cold, or icy cold. This “dynamic cool” gives a “wow” effect because it is strong and pleasant.

The “dynamic cool” sensation in the throat can be contrasted to the cool, cold, and icy cold sensations of rapid sipping of ice cold water or lemonade. For example: Take a glass of water equilibrated, (after stirring) with ice chips—a temperature of about 4° C. Start sipping the water at the rate of about 1 sip per second. The first 5 sips are pleasant, but by 5 to 10 sips, the throat feels a dull cold, and after about 10 to 15 sips, the icy cold in the throat becomes unpleasant, and the sensations of icy cold can be felt in the chest, half-way down to the stomach. These unpleasant sensations constitute “cold discomfort”.

Why are the sensations of sipping ice cream different from that of ice cold water? In both situations, the temperature of the contents in the throat is about the same, yet it is seldom possible to get unpleasantly cold in the throat with ice cream! One explanation is that the thermal conductivity of the oils and fats that make up ice cream is different from water. For example, the thermal conductivity value of olive oil is 0.17 W/m.K and that of water is 0.58 W/m.K. Ice water, with higher thermal conductivity (and higher thermal mass), abstracts more heat than ice cream. The rate of heat abstraction from the surface of the throat is then the determinant of the sensory perception and when it is too rapid or continuous, there is cold discomfort. On the other hand, a smooth heat abstraction rate produces a refreshing sensation. Experimentally, an ice cream with a high cream content, such as Haagen-Dazs® vanilla, works best for eliciting “dynamic cool”. The goal is then to identify a chemical sensory agent (i.e., a compound that does not abstract heat) that produces an optimal “dynamic cool” and not “cold discomfort”.

Surprisingly and unexpectedly, DAPA-2-5 elicits “dynamic cool” in the oropharynx for 5 to 15 min but without “cold discomfort”.

DAPA-2-5 elicits “dynamic cool” by action on receptive fields of afferents located in the orolaryngopharynx. The sensory nerves include the facial (7^{th})—innervating the surfaces adjacent to the palatine tonsils, the glossopharyngeal (9^{th})—innervating the posterior ⅓ of the tongue and walls of the oropharynx, and the vagus (10^{th})—innervating portions of the lateral/posterior walls of the oropharynx and the laryngopharynx. Further down the aerodigestive tract, the upper esophagus is innerved by the vagus and spinal afferents.

Technical difficulties prevent direct measurement of sensory inputs from the receptive fields of the 7th 9th and 10^{th} nerves, but mapping has been done for the 5th nerve, from receptive fields of the snout skin of rats. By inference, one can presume the processing of information is the same for all of these cranial nerves.

The central response of the 5th nerve neurons has been recorded and studied from rat superficial medullar dorsal horn that responds to innocuous thermal stimulation of the rat's face and tongue. Step changes of -A5° C. stimulated cells with both static firing rates and cells that had mainly dynamic properties [Davies, S N et al. Sensory processing in a thermal afferent pathway. J. Neurophysiol. 53: 429-434, 1985]. Similar studies in cats and humans showed that step decreases in temperatures (dynamic changes), as low as Δ 0.5° C./sec, were readily detectable by neurons and by psychophysical measurements [Davies, S N et al. Facial sensitivity to rates of temperature change: neurophysiological and psychophysical evidence from cats and humans. J. Physiol. 344: 161-175,1983],

From a study of the spike patterns of neuronal discharge (impulses/sec), it was clear that dynamic and not static firing responses to a change in temperature were the most powerful stimuli for generating coolness/cold sensations [see, e.g., Davies et al. 1983]. That is, the brain “sees” −Δ ° C./t and not absolute ° C. Thus, an agent that simulates optimal −Δ ° C./t on nerve discharge will produce “dynamic cooling”.

Method of Topical Delivery to Target

In this application, the concept for treatment is to topically apply an anti-nociceptive agent onto a portion of the receptive fields of the 5^{th}, 9^{th} and 10^{th} cranial nerves: for example, onto the surfaces of the oral cavity, oropharynx, upper esophagus and upper airways. The applied sensory agent is designed to counteract the effects of acid, irritants, and inflammation, and to relieve irritation, itch, and/or pain.

The fast transit time (˜35 cm/sec) of solids/liquids through the oropharynx is a hindrance to topical drug delivery to the neuronal receptive fields, but this obstacle can be circumvented by formulation of the active ingredient into an orally disintegrating tablet (ODT) placed on the mid-posterior dorsal surface of the tongue, or by focused delivery of the agent in liquid solution, e.g., as a macrodroplet, gel, or as an aerosol.

Rapid orally disintegrating tablets (ODTs) are defined as: “A solid dosage form containing medicinal substances, which disintegrates rapidly, within 30 sec, when placed upon the tongue. Furthermore, the products are designed to disintegrate or dissolve rapidly on contact with saliva, thus eliminating the need for chewing the tablet, swallowing an intact tablet, or taking the tablet with water” [S Department of Health and Human Service, Food and Drug Administration, Center for Drug Evaluation and Research (CDER). Guidance for Industry: Orally-disintegrating tablets, 2007].

Orally disintegrating tablets (ODTs) are normally used to deliver drugs into the bloodstream. In the context of the present discovery, ODTs are utilized as a method for localized topical delivery of an active ingredient onto the non-keratinizng stratified epithelium (NKSE) surface of the upper aerodigestive tract.

As contemplated for use in the present discovery, a tablet containing about 1 to 5 mg of DAPA-2-5, in a tablet weighing 50 to 150 mg (e.g., 0.6 to 10% of the tablet by weight) is sufficient for achieving the desired sensory effect. Rapid dissolution in the oropharynx is most effective if the irritation in the throat comes from nasal drip or acid reflux, but is less efficient if the irritants come from the airways up into the laryngopharynx. The dissolved contents of an oral tablet have greater difficulty coating the nerve endings at the entrance to the airways because swallowing initiates closure of the airway aperture. Spray delivery may be more efficient for the airway mucosa.

The DAPA compounds of this discovery are colorless or slightly yellow liquids at room temperature and have densities less than water, typically about 0.7 to 0.8 g/mL. There is superficial resemblance of DAPA molecules to C₅ to C₁₆ alkanes, but the phosphine oxide group has strong hydrogen bonding activity, and the effective compounds are soluble in water at concentrations of at least 10 to 20 mg/mL. By contrast, C₅ to C₁₆ alkanes do not mix with water. Nobody has ever tried before to produce an ODT with a DAPA compound using a tablet making machine, and this proved to be quite a challenge.

The creation of an ODT begins with the “granulation process” which is the collection of particles that will bond under compression to form the tablet. By choosing the right parameters, the recipe for a powder blend will be free-flowing into the funnel of the tablet press, be compactable to certain hardness, have minimal friablility, and fast disintegration. In recent years, direct compression is a favored method for making ODT wherein a group of ingredients are pre-blended, mixed with an active ingredient, placed onto a tablet press, and quickly made. Favored ingredients or excipients in the pre-blend are bulking agents such as polyhydric alcohols [e.g. mannitol or lactose] and lubricants such as magnesium stearate. Direct compression reduces the steps in manufacture and lowers costs. The blended powders of active ingredient and excipients are referred to as compressible direct-blend formulations.

Surprisingly, the mixing of DAPA compounds with a number of standard pre-blended formulations resulted in an oily non-free flowing powder. The exception was PharmaBurst 500 but this blend was not compressible. These formulation problems were overcome when DAPA-compounds were adsorbed onto a mineral excipient called magnesium aluminometasilicate. In a preferred embodiment of the discovery, the magnesium aluminometasilicate may be Neusilin™, or Al₂O₃.MgO.2SiO₂.xH₂O, produced by Fuji Chemical Industry Co., Ltd. [see www.fujichemusa.com].

An alternative to an ODT for oral administration is a gel, defined as a jelly-like material in a polymer mix that is non-flowing in its natural state. A hydrogel, that is, a gel in which water in a principal constitutent, is especially preferred as it will dissolve quickly at the back of the mouth. The use of gels or syrups as condiments is well known to the art and similar ingredients and formulations may be used for delivery of DAPA-2-5.

For liquid delivery, an alternative method is to use devices and dispensers charged with the active compound, and suitable for delivery of the active compound, for example:

to the oropharynx of a human;

to the upper esophageal surfaces of a human; and/or

to the upper airways of a human.

Preferably, the device or dispenser is a manually activated or metered-dose dispenser, with or without an adapter, to substantially selectively deliver the active compound onto surfaces of the human, for example, so that at least 70% by weight of the active compound by-passes the oral cavity and is delivered onto the intended surfaces. The delivered droplet may be an aerosol or a macrodroplet depending upon the aperture size and velocity of the dispensing mechanisms.

Preferably, the adaptor is a spacer attachment for the delivery device. Preferably, the spacer attachment has a length from 0.5 inch (˜1.27 cm) to 4.0 inches (˜10.2 cm). Preferably, the device or dispenser is adapted to deliver the active compound as a component of an aerosol or macrodroplet. Preferably, activation of the device or dispenser is adapted to deliver the active compound in a constant dose unit. Preferably, the total dose per activation period is 1 to 5 mg of the active compound. Preferably, the unit dose is derived from 0.05 to 0.2 mL of a liquid formulation of the active compound. Optionally, the device or dispenser is accompanied by instructions (e.g., written instructions) regarding its use.

The DAPA compounds, because of water solubility and chemical stability, are especially amenable to delivery as a focused aqueous spray or in fixed liquid aliquots or in syrups. These methods of liquid delivery may be useful for individuals who are unable easily to use solid dosage forms, e.g., young children, the elderly, and disabled individuals with difficulties in salivating or swallowing. The disadvantage of liquid formulations is rapid dispersion of the active ingredient onto the surfaces of the oral cavity or rapid transit of the liquid into the esophagus. This would limit contact time of the active ingredient to the intended target.

A preferred formulation is an orally-disintegrating tablet [ODT] containing 1 to 5 mg of DAPA-2-5. Such a formulation, when placed on top of the tongue at the back of the mouth, usually exerts a sensory effect in less than 10 sec and is effective for several hours for throat discomfort and heartburn. A preferred liquid formulation is 1 to 5 mg/mL of DAPA-2-5 dissolved in 25% (wt/vol) lemon juice, 1.5% (wt/vol) xylitol, and water. This solution can be placed in a plastic reservoir bottle and “squirted” onto the back of the mouth with a squeeze of the dispenser bottle. Alternatively, the solution may be place in a reservoir bottle with a manually activated spray pump with a spacer attachment of 3 inches (˜7.5 cm) that will facilitate delivery onto the surfaces at the back of the mouth.

The schedule of delivery of the agent is designed for an “as-needed” basis by the patient, and not as a fixed-interval drug. By this therapeutic strategy, the individual resumes voluntary control of upper aerodigestive discomfort, and can, for example, sleep better at night, gain peace of mind, and have less anxiety.

For the anogenital surfaces a cream, lotion, gel, solution, or a spray delivery system may be used.

Study 1

Toxicity

Preliminary toxicological studies were conducted on DAPA-2-5 and DAPA-2-7. Neither of these compounds was mutagenic in the Ames test (Strains TA 98 and TA100, with and without liver activation) (tests conducted by Apredica, Watertown, Mass., USA).

DAPA-2-7, dissolved in 3% ethanol/97% 1,2-propanediol, or vehicle alone, was administered subcutaneously to male rats (N=8 per group) at 30 mg/kg body weight daily for 7 days, and on the 8th day, the animals were euthanized with sodium pentobarbital and the major organs (body, heart, liver, lungs, kidney, testes, brain) were removed and weighed. Heart tissues (ventricle and heart valves) and liver samples were stained with hematoxylin and eosin and the histology examined. There were no significant differences in body or organ weights between the two groups and the heart and liver histology were normal.

A study with an identical design, but with DAPA-2-5, administered at 20 mg/kg perioral by gavage for 7 days (N=10 per group), gave similar results. In a large scale trial, DAPA-2-5 was administered via drinking water to male rats for 40 days. The DAPA-2-5 was mixed with condensed milk [8.5%] at 0.5 mg/mL and liquid consumption was monitored. The averaged intake in the treated group [N=12 rats] was 41 mg/kg body weight/day for 40 days. The vehicle alone group was N=11. In both experiments there were no statistical difference in organ weights or in histology between groups treated with DAPA-2-5 and groups treated with vehicle.

It was concluded that DAPA-2-5 could be orally sampled by human volunteers at a daily dose of less than 10 mg with minimal risks of adverse effects.

Study 2

Preparation of ODT for Sensory Testing on Oral Cavity and Pharyngeal Surfaces.

The DAPA molecules are liquids with a hydrophilic center attached to three hydrophobic alkyl chains, and thus create an unusual molecular environment for bonding to tablet excipients. Nobody has ever tried to create an ODT with the DAPA compounds of this discovery: and this objective became quite a technical challenge. In this discovery, an excipient of special interest is magnesium aluminometasilicate.

Three ODTs were made and labeled as ODT-A, ODT-B, and ODT-C. The details of formulation and preparation are described in some detail here because it was discovered that the granulation recipe is an “art form”, and that requires considerable efforts in experiment.

ODT-A was prepared by dissolving the DAPA compound in absolute ethanol, adding a 80% mannitol-20% maltitol mixture of equal weight and then adding an equal volume of distilled water. The mixture was the stirred with a glass rod, and dispensed onto a piece of wax or parchment paper with a disposable plastic pipette. The viscous liquid drop formed was then dried under a heat source for at least 4 hr. The nominal dose of the API in ODT-A ranged from 1 to 3 mg/tablet. The dried tablets weighed on average 150 mg each and when on the dorsal surface of the tongue, rapidly dissolved completely in saliva and coated the oropharyngeal surface. These tablets were suitable for initial pilot studies, but were not uniform in size, were friable, especially in conditions of high ambient humidity, and were not readily amenable to further analytical evaluation.

ODT-B was prepared by expert consultants at Formurex, Inc., 2470 N. Wilcox Road, Stockton, Calif. 95215. Tel: (209) 931 2040, under the supervision of Ravi Mahalingam, Ph.D. and Rajendra S. Tandale, Ph.D. This study is summarized in a report [FR-2013-1038, May 1, 2013, Formulation of DAPA-2-5 into Upper Esopharyngeal/Lower Pharynx Dosage Forms] incorporated here by reference. The experimental objectives were to develop prototype tablet formulations to deliver DAPA-2-5 at 1.5, 2.0, 5.0 and 8.0 mg doses, to characterize tablet properties, and to develop an HPLC method for quantifying release of -DAPA-2-5.

Experimental procedures. These materials, sources/Lot# were used: DAPA-2-5, Phoenix Pharmaceuticals, Inc. Lot #429773; Co- Processed Lactose, povidone and crospovidone (Ludipress), J. T. Baker Lot #1AH0516; Microcrystalline cellulose, NF (Avicel PH102), FMC Biopolymer Lot #P212824001; Co- Processed microcrystalline cellulose and guar gum (Avicel CE-15), FMC Biopolymer Lot # RH1082185; Povidone, USP (Plasdone K-29/32), ISP Technologies Lot #05100280713; Xanthan Gum, USP/NF (Xanthural 75). CP Kelco, Lot #2B4685K; Mannitol, USP (Mannogem), SPI Pharma, Lot #12000076G; Co-Processed ODT Excipient (F Melt), Fuji Chemical Ind. Co., Lot #201002; Maltodextrin NF altrin M510), Grain Processing Corporation, Lot # M0832960; Colloidal Silicon Dioxide (Cab-O-Sil M5P), Cabot, Lot #1222272; Aluminum Magnesium Metasilicate, USP (Neusilin US2), Fuji Chemical Ind. Co. Lot #009025; Co-processed ODT Excipient (Pharmaburst 500), SPI Pharma Lot #10M019; Co-processed Lactose (Ludipress), BASF Lot #17137275L0; Hypromellose, USP, 50 mPa.S, Spectrum Chemicals Lot #2B10277; Magnesium stearate, NF (HyQual)m Mallinckrodt Lot #20100125.

The pre-blended excipients supplied by manufacturers are generally bulking agents or diluents and sugars such as mannitol, lactose, or maltitol. Other excipients are microcrystalline cellulose, starch, colloidal silicon dioxide, sodium starch glycolate, and the lubricants are magnesium or calcium stearate. The study was conducted in three stages the DAPA Active Pharmacological Ingredient [API] was converted into a solid form by dilution with a pre-blended excipient, by 1) Dilution Approach, 2) Adsorption, or 3) Incorporation into mucoadhesive polymers.

Dilution Approach: DAPA-2-5 liquid were loaded onto excipients (batch size: 12-24 g) in ˜12 experiments. The API and the selected excipients were mixed geometrically using a stainless steel spatula, and the mixture passed through sieve #20. The sieved blend was lubricated using magnesium stearate (previously sieved through mesh #40), and evaluated for appearance and flow property (through funnel). Based on the flow nature, selected blends were compressed in to tablets using Ø7.2 mm (for 120 mg tablets) or Ø 9.5 mm (for higher than 120 mg tablet) standard concave round tooling on an Acura rotary press. Tablets were evaluated for thickness, hardness, friability, and disintegration test in 10 mL of purified water at room temperature. The appearance of powder blends and tablets were photographed.

The excipients: Ludipress, Avicel PH 102, Avicel CE-15, Mannitol, F-Melt, and Maltodextrin, yielded blends that were oily in appearance and had poor flow. The exception was Pharmaburst 500 blends. However, Pharmaburst 500 blends showed poor compressibility and varying the amount of Pharmaburst 500 did not enhance compressibility. Further tests were done with two silicate excipients: Neusilin US2, and Syloid 244FP. Surprisingly, Neusilin US2 was an effective excipient for blending with DAPA-2-5. Subsequent mixing with Pharmaburst yielded blends that could be compressed into tablets with acceptable physical properties of hardness, friability, and disintegration times. Various mucoadhesive polymers were tried and the results suggested that incorporation of hypromellose may have beneficial effects.

The formed tablet is evaluated by several parameters. The dissolution rate is the time for the tablet to dissolve in a fixed volume of water [e.g. 10 mL] and, in the case of the ODT, the disintegration time should be less than 30 sec. Tablet hardness measures the structural integrity of a tablet “under conditions of storage, transportation, and handling before usage”. Force is applied to the tablet until it breaks, and the unit of force can be in Newtons or a Kilopond (kp). The Newton unit is given by the equation 1 Newton=1 kg.m/s² where kg is kilogram, m is meter, and s is second. Friability, or being friable, describes the ability of a solid substance to break up to smaller pieces with little effort. A typical index of friability is the % of the tablet that is chipped off after mechanical stress.

The final preblend for ODT-B that might be optimal for clinical trials for a 100 mg ODT was a mix of DAPA-2-5: 1.5 mg, Neusilin US2: 20.0 mg, Blend: Pharmaburst 500 76.5 mg, Hypromellose [50 cps] 1.0 mg, and Mg- stearate: 1.0 mg, to give a non-oily free flowing powder that can be put into the funnel of a tablet making machine to yield tablets of Thickness (mm): 3.4, Hardness (kp): 3.0-5.0, Friability (%w/w): 0.04, Disintegration time (Sec): <20, and good Compressibility.

A high pressure liquid chromatography [HPLC] method was developed for analysis of DAPA-2-5 in aqueous solution. The chromatographic conditions were: Column: Waters XTerra RP18, 150×4.6 mm, 3.5 μm, Column temperature: 40° C., Mobile phase : 0.1% Tetrabutyl ammonium and Tri fluro acetic acid in 900mL water and 100 mL Acetonitrile, Flow: 1.0 mUmin, Diluent : Water, Wavelength: 226 nm, Injection Volume: 100 μL, Run time: 10 min, Standard Preparation: 0.75 mg/mL of DAPA-2-5 in water. Sample Preparation: Equivalent to 0.75 mg/mL of DAPA-2-5 in water. Prototype tablets containing DAPA-2-5 were disintegrated and the content of DAPA-2-5 measured. The results showed that 86±2% of DAPA-2-5 was recovered from the aqueous phase and thus available for bioactivity. It is likely that the non-eluted DAPA-2-5 remains tightly bound to sites on the Neusilin US2.

ODT-C. More experiments were conducted to find a smaller tablet, in the range of 40 to 60 mg, with a disintegration of <10 sec. These paraemters might achieve a better pharmacological effect. ODT-B was 100 mg in size and disintegrated in <20 sec. By trial and error it was found that a reproducible recipe for blending a DAPA ODT was:
a. a DAPA compound dissolved in an equal part of ethanol by volume, and then an 1.5 to 2.0 part by weight of Neusilin US2 is added, the combination mixed, and the ethanol is evaporated by placing the mixture under a heat source, e.g. a 100 watt light bulb. For example, to 1.5 g of DAPA-2-5 is added 1.5 g of absolute ethanol, followed by 2.0 g of Neusilin US2. The mixture is shaken or mixed in a Speedmixer at 500 rpm for 1 min. The sample is placed under a heat source and about 2 hr later, the sample size is re-weighed with the expectation that the ethanol has been evaporated and the sample now weights 3.5 g. This powder is free-flowing.
b. a sample of maltitol powder is prepared (Maltisorb® P200 from Roquette).
c. a sample of Prosolv® EasyTab is prepared (JRS Pharma, Rosenburg, Germany).
d. a blend of a. [2.80 g], b. [15.73 g] and c. [31.46 g] is prepared to make 1000 ODT tablets, each weighing 50 mg and containing 1.2 mg of DAPA-2-5.
e. the blend is first shaken by hand, then placed for 2 min in a SpeedMixer at 500 rpm. The blend is poured into the funnel of a New Single Punch Tablet Press Pill Making Machine Maker TDP-1-5 [available from Amazon.com] and the extruded tablets are collected.

A second inorganic excipient, CaHP04, Spray Dried Granule, Dibasic Calcium Phosphate Anhydrous; Calcium Hydrogen Phosphate, Anhydrous, trade name Fujicalin®, made by by Fuji Chemical Co., Ltd., was also found to be an effective adsorption ingredient for the DAPA compounds prior to blending with pre-mixed blens. The adsorption capacity was, however, half of Neusilin US2 and disintegration rate was twice as long.

In summary, prototype ODT formulations of DAPA compounds were created. The ODT were designed for delivery of the API onto the surfaces of the pharynx and esophagus. The granulation process was aimed at choosing the right parameters for a powder blend that will be free-flowing, compactable to certain hardness, and have minimal friability, and fast disintegration time. It was discovered that a key ingredient in successful formulation was the use of aluminum metasilicate oxide, commercially available as Neusilin US2. This excipient allowed the liquid DAPA compounds to be formulated into a tablet that met standard criteria for use. A representative formulation was composed of, DAPA-2-5 (1.5%), Neusilin US2 (20.0%), Pharmaburst 500 (76.5%), Hypromellose (1.0%), and Magnesium stearate (1.0%). An alternative pre-blend excipient to Pharmaburst 500 was EasySolv. These blends have excellent properties for compression into an ODT and met the criteria for compressiblility and ODT parameters.

Study 3

Sensory Qualities of ODT on the Oral Cavity and Pharyngeal Surfaces The DAPA ODT of Study 2 were evaluated for sensory properties on volunteers. The ODT-A results [see table] are from 6 to 8 trials per compound in several experienced individuals familiar with sensory properties of “cooling agents”. The results of ODT-A will be discussed together with ODT-B and ODT-C.

TABLE 5
Sensory activity of test compounds administered as ODT-A.

| | Onset | Duration | Sensory | Cold |
|---|---|---|---|---|
| Compound | (min) | (min) | Quality | Discomfort |
| DAPA-1-5 | 0.1 | 5 | cool | 0 |
| DAPA-1-6 | 0.6 | 16 | cool/cold | ++ |
| DAPA-1-7 | 0.6 | 16 | cool/cold | + |
| DAPA-1-8 | 1.2 | 27 | cool | + |
| DAPA-2-4 | 1 | 3 | brief cool | 0 |
| DAPA-2-5 | 0.1 | 12 | dynamic cool | 0 |
| DAPA-2-6 | 0.7 | 18 | cool/cold | ++ |
| DAPA-2-7 | 0.7 | 21 | cool/cold | ++ |
| DAPA-2-8 | 1.2 | 27 | cool | + |
| DAPA-3-1 | not active | 0 | not active | 0 |
| DAPA-3-2 | 1 | 2 | brief cool | 0 |

After application of ODT-A there were sensations associated with heat abstraction of various intensity and duration. Of particular note was the sensation of “icy or painful” cold was felt with some compounds, but the effect was variable. To isolate this sensation more precisely and reduce variability, ODT-A was placed on the tongue and 10 min later the subject was instructed to drink a mouthful of water previously equilibrated with ice chips. If the subject felt “normal coolness” in the throat, the value was “0”; if the water felt “excessively cold” on the throat, it was rated as “+”; and if there was “aching cold/discomfort” on the throat, the ranking was “++”. The term “cold discomfort” was applied compounds that produced “++”, and this phenomenon could now be reliably defined and identified with the cold water test.

ODT-B made by Formurex Corp. were pleasing in appearance, having uniform weight, hardness, color and geometry. After screening formulations, Compositions F25 [1.5 mg, 100 mg] and F27 [8 mg, 240 mg] were selected for detailed testing. The tablet size of 100 mg fits well on the tongue surface, but the complete dissolution in saliva was relatively slow of ˜1 min, despite a disintegration time of <20 sec in vitro, and a tablet hardness of 3 to 6 kp. The dissolution was accelerated by sipping a small quantity of water to wet the tongue, but this method of enhancing tablet performance may not be convenient in practice. F27, the larger tablet [8 mg of DAPA-2-5], worked well for the esophageal surface, and here the tablet can be taken with water. F27 cools the upper esophagus, and pleasant cooling can be felt in the chest, down to the level of the sternum. The F27 formulation and dose would be an excellent prototype for the treatment of heartburn and for the differential diagnosis of non-cardiac chest pain. The next formulation objective was to reduce tablet size and to hasten dissolution and delivery.

ODT-C, described in Study 2, were tested for sensory properties on 4 volunteers, with at least 3 trials per compound and at 1.2 to 1.5 mg per ODT. The ODT was placed on the mid-line posterior dorsal surface of the tongue and sensory effects recorded for 30 min on a standardized form and effects recorded for 30 min on a standardized form. The ODT were coded and administered on a double-blind basis. A modified ASHRAE scale of neutral [0], slightly cool [-1], cool [-2], cold [-3], and icy cold [-4] was used to rank the peak heat abstraction sensations in the mouth and throat. Other perceived effects, e.g. taste and duration, were also noted. The results are shown in Table 6.

TABLE 6
Sensory activity of test compounds administered as ODT-C.

| Code No. | ODT-C test results | ASHRAE equivalent |
|---|---|---|
| DAPA-1-5 | brief but intense cool, <5 min | −2.7 |
| DAPA-1-6 | intense sensation of icy cold, but has unpleasant | −3.5 |
| | taste on the tongue: test dose of 1 to 2 mg, long- | |
| | lasting >10 min | |
| DAPA-1-7 | intense sensation of icy cold, but has unpleasant | −3.5 |
| | taste on the tongue: test dose of 1 to 2 mg, >15 min. | |
| DAPA-1-8 | strong unpleasant taste, ~15 min | −3 |
| DAPA-1-9 | strong unpleasant taste, ~15 min | −2.7 |
| DAPA-2-4 | pleasant cool, but transient effect of <5 min | −2.5 |
| DAPA-2-5 | pleasant dynamic cool, ~12 to 15 min, at doses | −2.7 |
| | higher than 1.5 mg “penetrating cold” sensations. | |
| | Ideal for the treatment of sensory discomforts in | |
| | pharynx and esophagus | |
| DAPA-2-6 | intense sensation of icy cold, but has unpleasant | −3.2 |
| | taste on the tongue, long-lasting >15 min | |
| DAPA-2-7 | intense sensation of icy cold, but has unpleasant | −3.5 |
| | taste on the tongue, long-lasting >15 min | |
| DAPA-2-8 | strong unpleasant taste | −2.7 |

For all ODT-C with DAPA compounds, heat abstraction sensations were apparent within 1 min of placing the ODT. The onset was much faster than the ODT-B tablets, and may reflect the improved formulations. There was no ambiguity in the detection of heat abstraction sensations, so the use of placebo ODT, with only excipient but without a DAPA compound, was discontinued after 15 trials because these placebos were all recognized by the tests subjects and immediately ranked as zeros.

The heat abstraction sensations had qualitative differences in peak intensity or, in pharmacological terminology, maximal efficacy [or response], DAPA-2-5 produced refreshing coolness and cold, and when the dose was increased to 8 mg, there was a burning penetrating quality to the sensation, but it was not aversive: that is, there was no pain or overt discomfort. By contrast, 1-6, 1-7, 2-6, and 2-7 produced cold and icy cold which was painful to the back of the throat. The degree of pain and its duration was sufficient to rate these compounds as being undesirable for therapy.

DAPA-2-5 has a slight organic sweetish taste which was described by an old chemist as “chloroform-like”, By contrast, 1-6, 1-7, 2-6, and 2-7 and other analogs, e.g. 1-8 and 1-9, produced unpleasant taste described as “metallic”, “astringent”, “unripe persimmon-like”, and “harsh” which lasted for at least 20 min. The tastes had some resemblance to I-menthol at high doses of 8 to 10 mg on the tongue, but the “metallic” and other sensory qualities were different, and aversive: that is, the subjects said it was very unpleasant and undesirable. These unpleasant tastes were especially prominent with 1-8, 1-9, and 2-8. Attempts to mask the tastes with xylitol, an artificial non-caloric sweetener, were not successful. These differences in taste qualities were surprising, not predictable, and unexpected, and made DAPA-2-5 the best candidate for development.

The analogs 1-4 and 2-4 produced a cooling effect of <5 min and were therefore of no therapeutic value. DAPA-2-5 had a cooling effect which averaged ˜12.8 min, but the cooling duration varied with environmental conditions. The perception of cooling in the throat was more pronounced at night, near sleep time, presumably because there were fewer environmental cues for distraction. Thus, the heat abstraction sensations will persist for 20 min or longer instead of just 12 min. 2-7 had a longer cooling action than 2-6, 1-6 and 1-7. Although the overt cooling sensation may not be felt, the relief of sensory discomfort can persist for 3+hours.

From a structure-activity viewpoint, the results from ODT-A and ODT-C produced, surprisingly, closely similar results and conclusions, in spite of the difference in formulation. R₁ and R₂ must be secondary alkyl groups, that is, the carbon attached to (or alpha to) the phosphorus atom must be branched. Thus, the di-isobutyl moiety (in DAPA-3-1) is virtually inactive on oropharynx. Branching of the terminal carbon of R₃, wherein the n-pentyl of DAPA-2-5 is replaced by 3-methyl-butyl (DAPA-3-2), also results in significant loss of activity. But note that DAPA-2-5, DAPA-1-7, DAPA-3-1 and DAPA-3-2 have the same molecular weight, yet are distinctly different from each other in their activity. Active compounds are found in the di-isopropyl and di-sec-butyl series. Increasing the number of carbons in R₃ from C4 to C7 increased the duration of cooling, an effect that may be attributable to lipophilicity and tissue retention.

The unexpected and surprising observation here was DAPA-2-5 has “dynamic cool”. [when R₃ is n-pentyl]. But when R₃ is extended by one or two methylene groups to n-hexyl (DAPA-2-6) or n-heptyl (DAPA-2-7), the chemicals causes cold discomfort and adverse tastes. Reducing n-pentyl by one carbon to n-butyl (DAPA-2-4) retains cooling freshness, but this analog is too short-acting to be as useful in, for example, oropharyngeal disorders.

In summary, the ODT trials showed, surprisingly, that DAPA-2-5 is the best API for sensory discomfort in the pharynx, with a therapeutically effective duration of action. A second choice for an API is 2-7, but it is less ideal because of painful cold and bad tastes. There are qualitative differences of drug action that makes DAPA-2-5 selective and exceptional. The properties of the tested compounds, especially of adverse tastes, were unexpected and discovered only by further experiment and formulations.

The unique characteristics of the n-pentyl group in DAPA-2-5, with an absence of cold discomfort and adverse tastes, and localized dynamic cool, were surprising, and could not have been predicted from prior art. It was concluded that DAPA-2-5 is the best API candidate as an anti-nociceptive agent for the upper aerodigestive tract. DAPA-2-7 is an alternative if the unpleasant tastes are masked, and it has advantages of increased duration of action and an intense sensory experience.

Study 4

Sensory Quality on the Pharyngeal Surface: Pain

The “pain” component of inflammation includes irritation, itch, and discomfort and suppression of these endpoints is termed “anti-nociceptive”. To determine if a compound has anti-nociceptive activity, the Inventor has devised a modified capsaicin challenge method to evoke discomfort in the oropharyngx: the chili-pepper sauce irritation test.

Chili-pepper sauce placed onto the posterior dorsal surface of the tongue evokes a tickling/irritant sensations in the oropharynx. The sensations associated with the chili pepper sauce are located in the back of the mouth and are clearly recognized and associated with irritation and a desire to clear the throat.

Chili-pepper sauce applied onto the posterior dorsal surface of the tongue evoked sensations that can be readily suppressed with an ODT containing an active ingredient, but are not affected by an ODT containing only the excipient.

In the chili-pepper sauce irritation test, the test compound is delivered using an ODT, as in Study 3, and 30 min later 0.2 to 0.25 mL of the sauce was applied onto the posterior dorsal surface of the tongue (with a syringe or a plastic stick). The chili pepper sauce used here is called Yank Sing® Chili Pepper Sauce (YS Gourmet Productions, Inc., PO Box 26189, San Francisco, Calif. 94126) and is a well-known condiment for use with dim sum (Chinese tea lunch). If there was no suppression of the irritant/tickling sensations of the sauce, the value was “0”; if there was some suppression, the value was “+”; and if there was complete suppression, then the value was “++.” For compounds that gave a ++ score, the irritative signals of the chili-pepper sauce are completely absent, yet the salty taste from the soy sauce of the condiment can still be readily tasted.

The results are summarised in the following table. A “++” result indicated suppression of the irritant effects of chili-pepper sauce. Numerical results are from 6 to 8 trials per compound.

TABLE 7
Test compounds on sensitivity to a chili pepper
sauce.

| Compound | Chili test |
|---|---|
| DAPA-1-5 | ++ |
| DAPA-1-6 | ++ |
| DAPA-1-7 | ++ |
| DAPA-1-8 | + |
| DAPA-2-4 | 0 |
| DAPA-2-5 | ++ |
| DAPA-2-6 | ++ |
| DAPA-2-7 | ++ |
| DAPA-2-8 | + |
| DAPA-3-1 | 0 |
| DAPA-3-2 | 0 |

One unusual feature noted in the chili-pepper sauce irritation test was that the “dynamic cool” sensation on the oropharynx (measured in the pharyngeal irritation study) lasts about 10 to 15 min, whereas the anti-nociceptive activity (measured in the chili-pepper sauce irritation test) lasts for several hours. This “memory trace” action is most unusual and surprising, but may be explained if it is clearly recognized that clearance reflexes are essential to survival and, once evoked or enhanced, likely recruit other brain reflexes to cope with monitoring obstruction in the throat. This recruitment process in the brain may be long-lasting. The anti-nociceptive activity of the active DAPA compounds are quite intriguing and have not been previously observed.

Study 5

Agonist Activity on TRPM8, TRPV1, and TRPA1

The in vitro effects of test compounds were evaluated on cloned hTRPM8 channel (encoded by the human TRPM8 gene, expressed in CHO cells) using a Fluo-8 calcium kit and a Fluorescence Imaging Plate Reader (FLIPR^{TETRA™}) instrument. The assays were conducted by ChanTest Corporation (14656 Neo Parkway, Cleveland, Ohio 44128, USA).

Test compounds and positive control solutions were prepared by diluting stock solutions in a HEPES-buffered physiological saline (HBPS) solution. The test compound and control formulations were loaded in polypropylene or glass-lined 384-well plates, and placed into the FLIPR instrument (Molecular Devices Corporation, Union City, Calif., USA). The test compounds were evaluated at 4 or 8 concentrations with n=4 replicates per determination. The positive control reference compound was L-menthol, a known TRPM8 agonist. The test cells were Chinese Hamster Ovary (CHO) cells stably transfected with human TRPM8 cDNAs.

For the FLIPR^{TETRA™} assay, cells were plated in 384-well black wall, flat clear-bottom microtiter plates (Type: BD Biocoat Poly-D-Lysine Multiwell Cell Culture Plate) at approximately 30,000 cells per well. Cells were incubated at 37° C. overnight to reach a near confluent monolayer appropriate for use in a fluorescence assay. The test procedure was to remove the growth media and to add 40 μL of HBPS containing Fluo-8 for 30 min at 37° C. 10 μL of test compound, vehicle, or control solutions in HBPS were added to each well and read for 4 min.

Concentration-response data were analyzed via the FLIPR Control software that is supplied with the FLIPR System (MDS-AT) and fitted to a Hill equation of the following form:

$$RESPONSE = Base + \frac{Max - Base}{1 + {( \frac{xhalf}{x} )}^{rate}}$$

where: “Base” is the response at low concentrations of test compound; “Max” is the maximum response at high concentrations; “xhalf” is the EC₅₀, the concentration of test compound producing half-maximal activation; and “rate” is the Hill coefficient. Nonlinear least squares fits were made assuming a simple one-to-one binding model. The 95% Confidence Interval was obtained using the GraphPad Prism 6 software.

The results (agonist activity in the TRPM8 receptor assay) are summarized in the following table.

Of the 11 DAPA compounds tested, all showed full efficacy on the TRPM8 receptor, i.e. at the highest tested concentration there was ˜100% stimulation of calcium entry, and the data fitted a sigmoidal dose-response curve. The EC₅₀ of the more potent sensory compounds 1-6 to 1-8, and 2-5 to 2-8 fell within a narrow range with overlapping 95% Confidence Intervals. There were no distinguishing features in the EC₅₀ which enabled prediction of the compounds with desired cooling properties in the upper digestive tract. The structural modifications of 3-1 and 3-2 resulted in a significant loss of bioactivity.

TABLE 8
TRPM8 agonist activity of test compounds.

| | | 95% | Relative |
|---|---|---|---|
| | | Confidence | Potency |
| Compound | EC₅₀ (μM) | Interval | to L-menthol |
| Menthol | 3.8 | 2.5 to 5.6 | 1.0 |
| DAPA-1-5 | 5.6 | 4.4 to 7.2 | 0.7 |
| DAPA-1-6 | 2.4 | 1.5 to 4.0 | 1.6 |
| DAPA-1-7 | 0.7 | 0.5 to 1.0 | 5.4 |
| DAPA-1-8 | 0.7 | 0.5 to 1.0 | 5.4 |
| DAPA-2-4 | 14.5 | 7 to 29 | 0.3 |
| DAPA-2-5 | 1.7 | 1.0 to 2.9 | 2.2 |
| DAPA-2-6 | 0.8 | 0.5 to 1.3 | 4.7 |
| DAPA-2-7 | 1.1 | 0.6 to 2.3 | 3.4 |
| DAPA-2-8 | 1.3 | 0.7 to 2.3 | 2.9 |
| DAPA-3-1 | 24 | 8 to 76 | 0.2 |
| DAPA-3-2 | 4.2 | 1.6 to 10.8 | 0.9 |

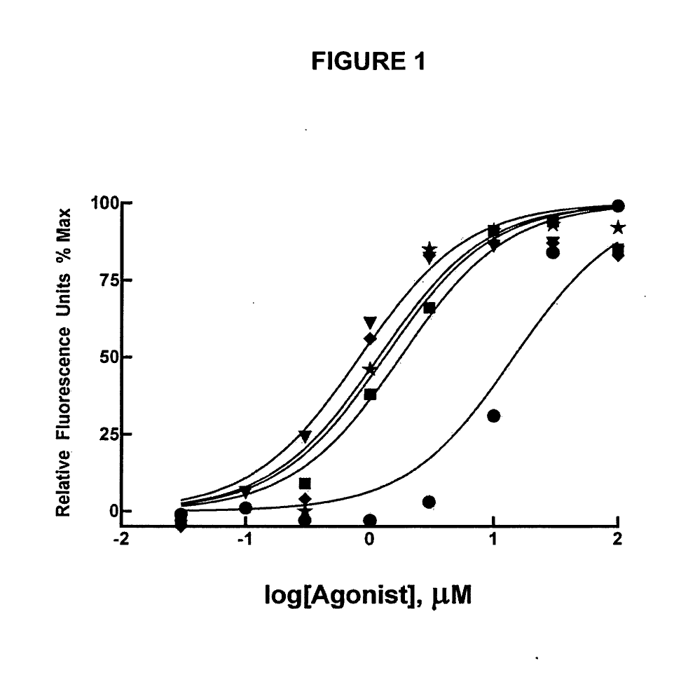

The results for the DAPA-2 series are shown in FIG. 1.

FIG. 1 is a graph of fluorescence (Relative Fluourescence Units; % Maximum) of test compounds evoked in TRPM8 transfected cells as a function of the logarithm of the concentration of the test compound (μM), for each of DAPA-2-4 (circle), DAPA-2-5 (square), DAPA-2-6 (inverted triangle), DAPA-2-7 (diamond), and DAPA-2-8 (star).

DAPA-2-4 is significantly less potent than DAPA-2-5, DAPA-2-6, DAPA-2-7, and DAPA-2-8. The potencies of DAPA-2-5 to DAPA-2-8 were similar with overlapping 95% confidence intervals. Nevertheless, DAPA-2-5 is preferred because there are distinct, selective pharmacological differences among these compounds when administered in vivo.

To examine the specificity of the test compounds, further studies were conducted on TRPV1 channels (human TRPV1 gene expressed in HEK293 cells) and TRPA1 channels (human TRPA1 gene expressed in CHO cells). The test cells were Chinese Hamster Ovary (CHO) cells or Human Embyronic Kidney (HEK) 293 cells transfected with human TRPV1 or TRPA1 cDNAs. The positive control reference compound was capsaicin (a known TRPV1 agonist) or mustard oil (a known TRPA1 agonist).

DAPA-2-5, DAPA-2-6, and DAPA-2-7 did not exhibit any agonist activity on TRPA1 channels at maximum tested concentrations of 100 μM.

Surprisingly, DAPA-2-5, DAPA-2-6, and DAPA-2-7 exhibited a weak TRPV1 agonist activity with projected EC₅₀ of 7.0 mM, 0.13 mM, and 0.22 mM, respectively. Note that DAPA-2-5 is 54 times less potent than DAPA-2-6 in stimulating TRPV1. The relative potencies of DAPA-2-5, DAPA-2-6, and DAPA-2-7 were confirmed in a second experiment, and may provide a basis for the different pharmacological properties observed with these compounds.

DAPA-2-5 was also evaluated at 5 μM in patch-clamp experiments in cells transfected and expressing channel receptors for ASIC3 (acid-sensing), hNav1.7 (sodium channels), and hERG (potassium channels). No agonist or antagonist activities were observed for DAPA-2-5 in these cells, although the positive controls (i.e., amiloride, lidocaine, and E4031) were active in these cells, respectively.

In summary, the TRPM8 EC₅₀ [median effective dose] seems to have little predictive value. The 95% Confidence Limits of many EC₅₀ overlap and only analogs with at least a 5-fold difference in potency are clearly distinguishable from each other. From the ODT studies, it was clear that “dynamic cool”, icy cold, adverse tastes, and duration of action were important parameters. But the EC₅₀ does not give information on the quality of the heat abstraction sensation, the likelihood of unpleasant taste, or the duration of drug effect. Thus, accessibility to and efficacy at TRPM8 are not defined by the EC₅₀. To over-interpret the EC₅₀ is naïve. Other bioassays are required to address the questions of selectivity.

Study 6

Suppression of Heat-Induced Edema in an Animal Model

Inflammation is defined as the reaction of vascularized living tissue to local injury [Cotran, RS. 1989. Inflammation and repair. In Robbins: Pathologic Basis of Disease, ed. S. L. Robbins, R. S. Cotran, V. Kumar, 2:39-86. Philadelphia: Saunders. 4^{th} ed.]. The characteristic signs of inflammation are redness, swelling, heat, and pain (and loss of function).

The anti-inflammatory properties of the DAPA compounds were studied in a model of heat-induced vascular leakage [Wei et al. Method of inhibiting inflammatory response. U.S. Pat. No. 4,801,612; Wei et al., Anti-inflammatory peptide agonists. Annual Review Pharmacol Toxicol. 33:9-108, 1993]. When the paws of pentobarbital-anesthetized rats (200 to 300 g body weight) are immersed in 58° C. water for 1 min, the normal paw volume of about 1.8 mL was increased by ˜88% within 30 min, the swelling being due to an increase in water content of the paw. The test was to see if the paw is exposed for 30 min before heat to selected DAPA compounds reduced the heat-induced increases in paw volume.

Test compounds were dissolved in 20% water-80% R-1,2-propanediol at 20 mg/mL. The solutions were applied to the paw skin of pentobarbital-anesthetized rats (200 to 300 g body weight) at 0.3 mL per paw, using a syringe attached to a blunt 21 gauge needle covered at its tip with a piece of polyethylene 60 tubing. After distributing the solution over the paw, the paw was tightly enclosed in a plastic finger cut from a disposable glove. The contralateral paw [control] received only the vehicle. Thirty min after application, both paws were immersed in 58° C. water for 1 min. Thirty min after immersion, both paws were cut at ankle joint with scissors and weighed.

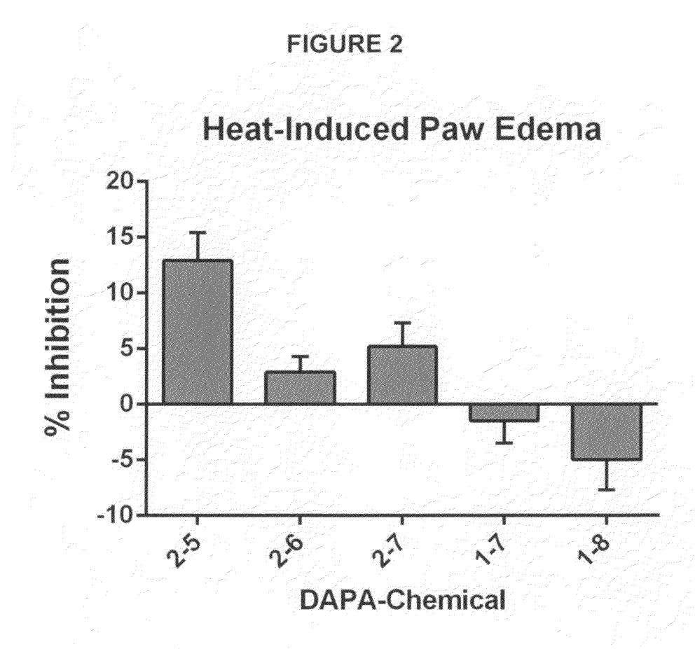

In preliminary studies, it was found that the paws of control animals (N=12) increased in weight from 1.77±0.02 g (mean±S.E.M.) to 3.33±0.07 g after heat exposure (58° C. water for 1 min), an increase in paw weights of 88±2%. As shown by the data in FIG. 2, DAPA-2-5 significantly inhibited this response, relative to the contralateral paw, by 12.9% (P<0.01). This degree of inhibition is significant for tissue protection as the injury stimulus is of supra-maximal intensity. The DAPA-2-5 effect was apparent to the untrained observer. These results were surprising because this anti-inflammatory property of DAPA-2-5 was not expected, and had not been previously reported in the scientific literature; furthermore, the inhibitory effects of DAPA-2-5 were not seen for the other DAPA compounds.

The mechanisms of heat-induced edema have been discussed elsewhere [Reed et al., Transcapillary exchange: role and importance of the interstitial fluid pressure and the extracellular matrix. Cardiovascular Res. 87: 211-217, 2010]. The denatured proteins of the heat-injured tissues unfurl and expose hydrophilic groups. The decreased interstitial fluid pressure of the extracellular matrix then imbibes water from the blood compartment into the skin. This results in rapid tissue edema. DAPA-2-5 inhibits this process but not the other tested DAPA compounds [see FIG. 2].

TABLE 9
Effects of test compounds on heat edema.

| | | Inhibition of Heat Edema |
|---|---|---|
| Compound | Mol. Wt. | (±S.E.M.) |
| DAPA-2-5 | 232 | 12.9 ± 2.5* |
| DAPA-2-6 | 246 | 2.9 ± 1.4 |
| DAPA-2-7 | 260 | 5.2 ± 2.1 |
| DAPA-1-7 | 232 | −1.5 ± 2.0 |
| DAPA-1-8 | 246 | −5.0 ± 2.7 |

*P < 0.01 vs contralateral paw

Study 7

Inflammatory Effects in an Animal Model

The icy-cold stinging sensations observed with some of the DAPA compounds suggested that they might be direct irritants. This hypothesis was tested by applying 20 μL of the pure compound onto the shaved abdominal skin of a pentobarbital-anesthetized rat. The test substance was enclosed in a circle of ˜1 cm diameter with a ring of cream. The test substance was applied with a micropipette, and after 1 hour the area was wiped with a cotton pad and the presence of redness (irritation) was graded on a scale of 0 to +++.

The data are summarised in the following table.

TABLE 10
Irritant activity of test compounds on rat skin.

| | | Redness after 20 μL applied topically |
|---|---|---|
| Compound | Mol. Wt. | to skin (scale of 0 to +++) |
| DAPA-2-5 | 232 | 0 |
| DAPA-2-6 | 246 | + |
| DAPA-2-7 | 260 | ++ |

The data show that each of DAPA-2-6 and DAPA-2-7, in undiluted form, is a skin irritant. By contrast, pure DAPA-2-5 has minimal or no pro-inflammatory actions on the skin.

The lack of inflammatory actions of DAPA-2-5 is important as some of the intended uses of DAPA-2-5 are on inflamed mucous membranes and transitional epithelia, and any nociceptive actions of DAPA-2-5 may exacerbate irritancy or pain. The exact reasons for selectivity and non-selectivity of these structurally-similar compounds are not clear at this time, but may involve interactions at other receptors such as TRP channels such as TRPV1, which are activated by DAPA-2-6 and DAPA-2-7 at high concentrations.

These results also have special relevance to the possible use of DAPA-2-5 in the pharyngeal disorder known as laryngopharyngeal reflux (LPR). In LPR, stomach acid and pepsin are regurgitated onto the laryngopharyngeal surfaces and causes tissue injury. The inflammation around the larynx is readily visualized in the patient with a laryngoscope, and the inflammation causes pain, hoarseness, and throat clearing. Currently, the primary method of treatment is to reduce acid secretion from the stomach, for example, with the use of proton-pump inhibitors; however, there are no methods to treat the discomfort in the throat or the inflammation of the pharyngeal mucosa. An agent such as DAPA-2-5, formulated for delivery as an orally disintegrating tablet (ODT), gel, liquid solution, or aerosol, offers a novel strategy for therapy of the inflamed mucous membranes of LPR.

Study 8

A principal endogenous irritant in the linings of the upper aerodigestive tract is hydrochloric acid. Acid stimulations of the mucosa of the pharynx will elicit reflex swallowing. Receptive regions are in the pharyngeal walls and innervated by the glossopharyngeal nerve (9^{th}) and the interior superior laryngeal nerve (10^{th}). In a rat animal model, solutions of organic acids such as acetic acid and citric acid were effective in eliciting swallowing [Kajii et al., Sour taste stimulation facilitates reflex swallowing from the pharynx and larynx in the rat Physiology & Behavior 77: 321-325, 2002]. In the present discovery, the method for measuring sensory responses to acid was adapted for screening agents that might suppress the sensitivity to hydrochloric acid. Agents that suppress the acid challenge may then have utility in relieving the discomfort of heartburn.

Experiments were conducted at the Pavlov Institute of Physiology, St. Petersburg, Russia. Adult male Wistar rats, weighing 200 to 400 g, were obtained from the Roppolovo Vivarium. All rats were anesthetized with sodium pentobarbital/urethane and fixed in the supine position. Body temperature was maintained at 37.0° C. with a heating pad. A midline incision was made in the ventral surface of the neck and the trachea was cannulated. A guide tube (polyethylene, diameter 2.2 mm) was fixed at the midline of the hard palate and an internal infusion tube (polyethylene, diameter 0.9 mm) was then placed flush to the end of soft palate. This procedure allows stimulation of the pharyngolaryngeal region with liquids, with minimum mechanical perturbation. An esophageal tube was placed at the thoracic level to drain solutions after infusion. The infused solution was applied to the pharyngolaryngeal region at a flow rate of 1.5 pUs for 20 sec using an infusion pump, giving a total unit volume of approximately 30 μL. Stimulations were applied at intervals of 2 to 3 min, with intervals allowed for rinsing and cleansing with suction. The solutions infused were distilled water, normal saline, 0.1 N hydrochloric acid, or test compounds dissolved in normal saline. A paired unipolar electrode was inserted into unilateral mylohyoid muscle to record electromyogram (EMG) activity and the signal processed for later analysis. Swallowing movements was identified as the EMG activity and could also be visualized as laryngeal movement. The number of swallows in a fixed interval was used as the endpoint.

The test procedures were similar to those described earlier (see, e.g., Kajii et al., 2002). Also, the drainage of the esophagus was at the thoracic level to avoid mechanical disturbance of the pharynx. The infusion rate of solutions was 1.5 μL/sec for 15 to 18 sec.

Saline, the vehicle for test compounds, did not induce swallowing movements. The response to 0.1 N HCl infusion (30 μL over an approximately 18 second period) was highly reproducible. On average there were 36±4 swallowing movements within one min (N=7 rats). This response could be elicited in the same rat over a 2 hour period with a rest period or saline infusions at 10 min intervals.

The up-down method of Dixon [Efficient analysis of experimental observations. Ann. Rev. Pharmacol. Toxicol.: 20, 441-462,1980] was used to titrate inhibition of the swallowing response and obtain an EC₅₀ with 50% reduction of swallowing frequency as an end-point for a quantal response. The EC₅₀ (N=8) for DAPA-2-5 was estimated to be 0.09 mg/mL.

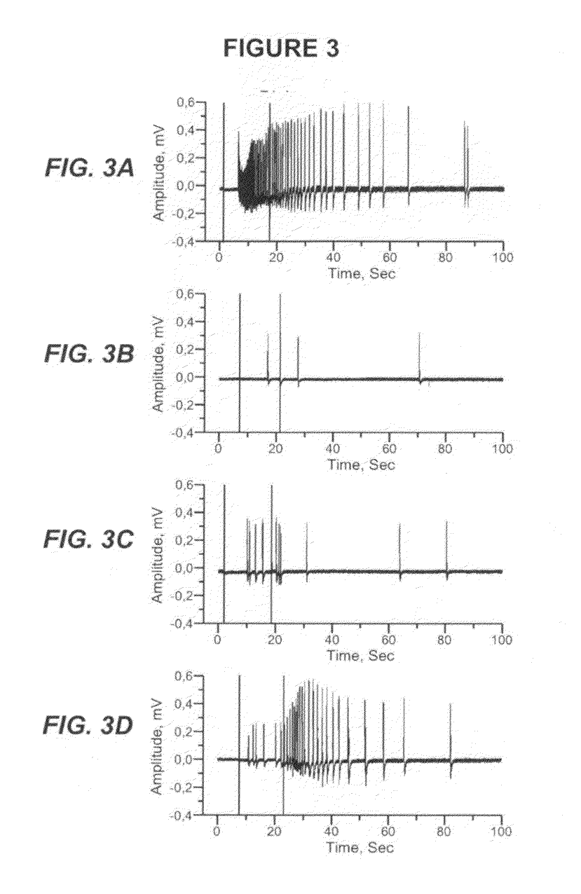

An example of an experiment is shown in FIG. 3.

FIG. 3 shows graphs of the amplitude (mV) of electromyogram (EMG) activity recorded from the myohyloid muscle of the anesthetized rat after infusion of 0.1 N HCl into the oropharynx. FIG. 3A: 47 swallows after acid. FIG. 3B: Infusion of DAPA-2-5 (0.4 mg/mL at 1.5 μL/sec) for approximately 18 sec inhibited the acid challenge given 5 min later (3 swallows/min). FIG. 3C: A second acid challenge given 10 min after DAPA-2-5 elicited only 9 swallows per min. FIG. 3D: After a saline rinse (1.5 μL/sec for 20 sec), a third acid challenge, 15 min after DAPA-2-5, gave the partially restored response of 27 swallows/min.

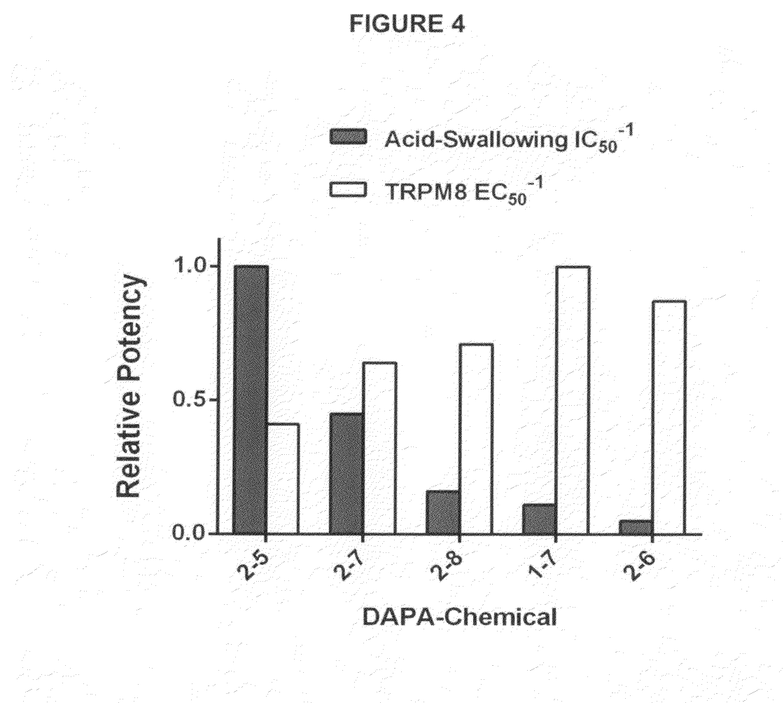

The data for all tested compounds are summarized in the following table (with N=4 to 8 experiments per compound). For comparison, the EC₅₀ for the TRPM8 receptor assay is shown in the last column. DAPA-2-5 was the most potent compound for suppression of acid-induced swallowing, even though DAPA-2-6, DAPA-2-7, and DAPA-1-7 were more potent in the TRPM8 receptor assay. The comparison of the relative potency for EC₅₀ and IC₅₀ of the tested DAPA compounds are shown in FIG. 4. It can be clearly seen that the two bioassay measures of potency are not correlated.

TABLE 11
Potency of various analogs for the suppression of acid-
induced swallowing movements in the anesthetized rat

| | | | | TRPM8 |
|---|---|---|---|---|
| | EC₅₀ ± S.E.M. | Relative | | Relative |
| Code | (mg/mL) | Potency | Comments | Potency |
| DAPA-2-5 | 0.09 ± 0.02 | 1.0 | reversible; | 0.41 |
| | | | ~15 min | |
| DAPA-2-7 | 0.20 ± 0.02 | 0.45 | long-lasting; | 0.64 |
| | | | >~30 min | |
| DAPA-2-8 | 0.56 ± 0.17 | 0.16 | — | 0.71 |
| DAPA-1-7 | 0.82 ± 0.08 | 0.11 | — | 1 |
| DAPA-2-6 | 1.7 ± 0.7 | 0.05 | — | 0.87 |

The lack of correlation in potency between the TRPM8 EC₅₀ assay and IC₅₀ for inhibition of acid-induced swallowing is quite striking. For example, DAPA-2-6 has twice the potency of DAPA-2-5 in the receptor assay, but only 5% of its inhibitory activity for swallowing. DAPA-2-7 is less potent than DAPA-2-5, but its duration of inhibition on swallowing is more long-lasting. For the differential diagnosis of chest pain, DAPA-2-7 may be more efficacious than DAPA-2-5 because of its longer-acting effects.

These experimental results again emphasize the unusual and selective activity of DAPA-2-5 on the upper digestive tract, relative to similar analogs. The results in this animal model of acid irritation are especially relevant for the potential use of DAPA-2-5 in suppressing acid-reflux related discomfort of the pharynx and upper esophagus. It should be noted, however, that the longer duration of action DAPA-2-7 versus DAPA-2-5 suggests that it may be an alternative candidate to DAPA-2-5 for use in acid-reflux diseases.

Study 9

Studies on Isolated Vaqus Nerve: Direct Anti-nociceptive Activity

To examine the ability of DAPA-2-5 to suppress sensory discomfort, it was tested in an animal model developed at the Imperial College, London, U.K. [Birrell et al. TRPA1 agonists evoke coughing in guinea pig and human volunteers. Amer. J. Respiratory and Critical Care Medicine 180, 1042-7, 2009; Patel et al., Inhibition of guinea-pig and human sensory nerve activity and the cough reflex in guinea-pigs by cannabinoid (CB2) receptor activation. British J. Pharmacol. 140, 261-8 2003]. In this in vitro assay, segments of the vagus nerve are placed on a platform and the electrical activity is recorded after topical application of capsaicin. Capsaicin is a known irritant that elicits pain when it is applied to the skin and it will depolarize the isolated vagus. The ability of substances to inhibit this capsaicin-induced depolarization is measured.

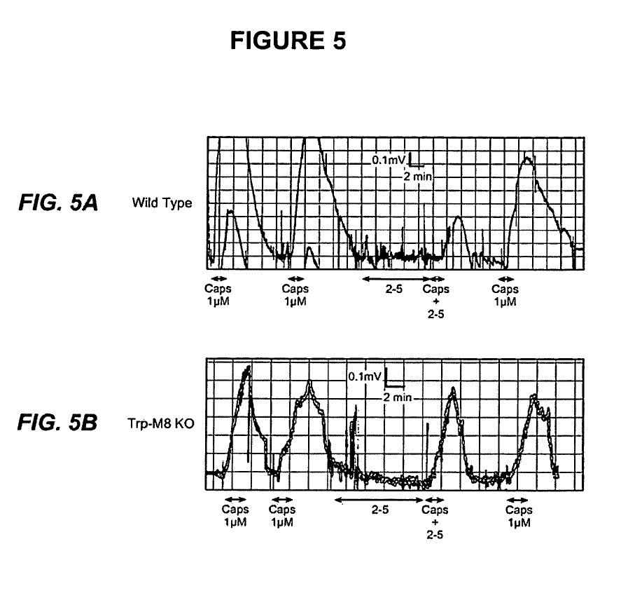

Briefly, segments of vagus nerve, caudal to the nodose ganglion, were removed from mice with fine forceps and segments placed in oxygenated Krebs solution and bubbled with 95% O₂/5% CO₂. The desheathed nerve trunk was mounted in a ‘grease-gap’ recording chamber and constantly superfused with Krebs solution with a flow rate of approximately 2 mL/min, and the electrical activity of the nerve monitored with electrodes. The temperature of the perfusate was kept constant at 37° C. by a water bath. Nerve depolarizations were induced by superfusion of the nerve with capsaicin (1 μM). After two reproducible depolarization responses to capsaicin, DAPA-2-5 was applied at 1 mg/mL (4 μM) for 10 min in the perfusate followed by capsaicin. The nerves were then washed with Krebs until the responses had returned to baseline and challenged again with capsaicin. The results and tracings obtained in normal and Trp M8 knockout mouse are shown in FIG. 5.

FIG. 5 shows polarization traces that illustrate, in the first trace FIG. 5A (“Wild Type”), the inhibition of capsaicin-induced depolarization of the isolated mouse vagus by DAPA-2-5, superfused at a 1 mg/mL, and, in the second trace FIG. 5B (“TRPM8 KO”), the significant absence of inhibition in the isolated TRPM8 KO mouse vagus by DAPA-2-5, superfused at a 1 mg/mL.

In the tracings shown in FIG. 5, the first two peaks show the depolarization response of the mouse vagus to capsaicin (“Caps”). After DAPA-2-5 is applied (1 mg/mL), the response is suppressed in the normal mouse vagus FIG. 5 A (“Wild Type”), but not in the TRPM8 knock-out FIG. 5 B (“TRPM8 KO”) mouse vagus.

The per cent inhibition of capsaicin-induced depolarization of the isolated normal mouse vagus caused by DAPA-2-5 was about 60%; the per cent inhibition of capsaicin-induced depolarization of the isolated TRPM8 knock-out mouse vagus caused by DAPA-2-5 was about 0%.

This experiment clearly demonstrates a direct pharmacological action of the DAPA-2-5 on the sensory nerve, which is a surprising and unexpected result. Furthermore, the diminished response in the TRPM8 KO mouse indicated that the receptor target was TRPM8. These results provide strong evidence that DAPA-2-5 can be used as an anti-nociceptive agent on the non-keratinizng membranes innervated by a sensory nerve such as the vagus.

Capsaicin is a TRPV1 agonist and the search for an effective TRPV1 antagonist has been the super-intense quest of many pharmaceutical companies for the past ten or more year. Here, it is shown that DAPA-2-5 is an effective “physiological” antagonist of TRPV1 at low concentrations. DAPA-2-5, by itself, did not evoke depolarization, indicating that it is free of agonist activity at this “pain” receptor. These results strongly indicate the usefulness of DAPA-2-5 an anti-nociceptive agent.

Study 10

Guniea Pig Cough Model

This study was conducted at the State Key Laboratory of Respiratory Disease, Guangzhou Institute of Respiratory Disease, Guangzhou Medical University, Guangzhou, China The investigators were Dong PeiJian, Liu ChunLi, Zhang Qingling, Wei TakFung, and Zong NanShan.

DAPA-2-5 was evaluated in a standardized guinea pig model of cough. Male specific pathogen—free guinea pigs about one month of age, weighing 250 to 300 g, were obtained from the medical experimental animal center of Guang Dong Province, China, and housed in rooms maintained at 22±2° C. and 55±15% humidity with a 12-hr light cycle and 20 air changes per hr. The experimental procedures were approved by the Institutional Animal Care and Use Committee.

Guinea pigs were placed in a transparent plastic 2 L plethysmography chamber equipped with a bias flow regulator that withdrew air at a rate of 2.5 L/min (Buxco, Wilmington, N.C., USA). Two mL of a 1 M citric acid (Sigma) dissolved in saline, was delivered into the chamber using an ultrasonic nebulizer with an output of 0.6 mL/min, delivering an aerosol with an estimated median particle diameter of 0.9 mm (Aeroneb® Lab Nebulizer System, Aerogen, Galway, Ireland). Animals were exposed for 10 min.

Cough in the guinea pig occurs as a rapid, abdominal movement, accompanied by a characteristic vocalization. The frequency was detected as a transient change in airflow in the chamber and the signal recorded via a pressure transducer and computer. Additionally, the audio-amplified count was also recorded electronically. Coughs were counted for the 10 min exposure period. The experiment was visually monitored by the investigator.

The baseline frequency of coughing was recorded for all animals (N=20) and 7 days later, animals were anesthetized with diethyl ether. A small animal laryngoscope was used to place the tip of a microsprayer syringe in the oral cavity. The specialized instruments for small dose delivery were from Penn Century Inc. Wyndmoor, Pa. 19038 USA. Saline, or DAPA-2-5 dissolved 2 mg/mL in saline, at N=10 per group was administered at 75 μL per animal. Ten min after injection, the guinea pigs were exposed to the citric acid mist and the number of coughs recorded.

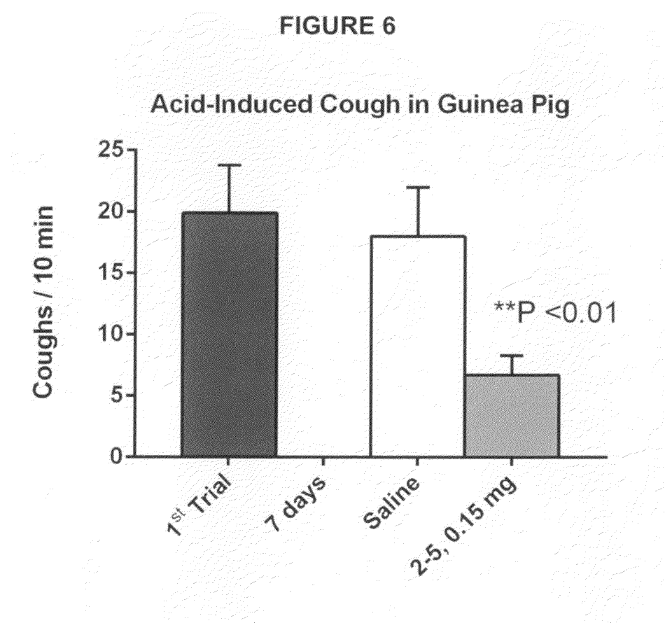

FIG. 6 shows a graph of the results. Guinea pigs (N=20) exposed to 1 M citric acid solution had a cough frequency of 19.9±3.9 coughs per 10 min observation period. One week later, saline delivered into the oropharyngeal region did not influence cough frequency (18.0±4.0 coughs), but DAPA-2-5, 2 mg/mL in saline, delivered at 75 μL per animal, inhibited cough (6.7±1.6 coughs, P<0.01 Mann-Whitney U test). These preclinical studies in the anesthetized rat and in the guinea pig establish that DAPA-2-5 and related molecules have anti-nociceptive actions against acid stimuli in the upper digestive tract.

This study was repeated with an identical design, but using capsaicin 0.1 mM mist as the stimulus. DAPA-2-5 also inhibited the capsaiscin-induced coughing in this guinea pig model. This is the first instance in which a topical cooling agent has been shown to inhibit cough in a standardized animal test model.

Case Study 1

A 62-year old male was a senior executive at a pharmaceutical company. He had a busy work schedule but was susceptible to viral colds which resulted in a persistent hoarseness and cough. These symptoms were difficult for him socially because he liked to attend opera and it also interfered with his persona at constant business meetings which lasted for several hours and which required his active participation. He volunteered to try orally disintegrating tablets (ODT-A) containing 1 to 1.5 mg of DAPA-2-5. These ODTs were prepared using mannitol (75 to 80% wt/wt) and maltitol or xylitol (20 to 25% wt/wt) as the excipients. The tablets generally weighed from 50 to 120 mg each and contained from 1 to 5 mg of DAPA-2-5. For this individual, the dose of 1 to 1.5 mg of DAPA-2-5 was fully effective in relieving his hoarseness on five occasions when he needed it. He remarked that the convenient size of the ODTs allowed him to take tablets with discretion. He remarked that DAPA-2-5 ODTs had fast onset and a “smooth” feel. He said that the ODT, if not prepared properly, sometimes had an “edgy” feel and tickled the throat and caused coughing before the onset of “dynamic cool”, but otherwise there were no side effects. He also tried solutions of 2 mg/mL DAPA-2-5 squirted onto the back of his throat and said that these felt fine. He declared that this was the best medication he had ever taken for an uncomfortable throat.

Case Study 2

A 70-year old retired architect had a viral cold for 3 weeks and could not sleep well because of severe nasal stuffiness. He could not breathe through his nose and lying down on the bed exacerbated his sense of frustration. He volunteered to try the tablets containing DAPA-2-5. Within a min of taking a tablet, he said he felt the “wow” effect in the back of his throat and could swallow some of the accumulated materials in his nose. He said there was a suction effect which helped him clear his nose and allowed him to breathe better. He took tablets for three successive nights and said he slept better than he had in the three preceding weeks. He remarked that two tablets each containing 1 mg of DAPA-2-5 or one tablet containing 1.5 mg of DAPA-2-5 was an effective remedy for his nasal congestion. This result was surprising because it indicated that discomfort of the nasal membranes, including the nasopharynx, could be relieved by the sensory effects of DAPA-2-5 in the throat. It is possible that the DAPA-2-5 had a vasoconstrictive action on the pharyngeal mucosa and reduced airflow resistance in the nasopharynx.

Case Study 3

A 68-year old male was on vacation in Carmel-by-the-Sea in California. During dinner, he drank two glasses of wine. His nasal passages felt somewhat congested because he was allergic to the flowers in bloom. In the evening, he took an orally disintegrating tablet (ODT) containing 2 mg of DAPA-2-5 and, lying in the bed, he said the airflow in his nose and throat felt soothing and “super-comfortable”. The cool air, with a tinge of the sea, was perfect. There was no resistance to flow, and his breathing was “effortless”. He was euphoric and ecstatic over this experience.

Case Study 4

Two subjects, a 66-year old female and a 69-year old male, had asthma. They both had bouts of dry and wet coughs during the day which they said could be tolerated. At night, however, they frequently woke up in the middle night with a sense of choking and accumulated materials in the throat. They remarked on the wheezing as airflow in the chest encountered the accumulated phlegm. In both individuals, orally disintegrating tablets (ODT-A) containing 1 mg DAPA-2-5 were effective in controlling choking and gagging and allowed them to fall asleep again. One subject wrote in an email: “Just want you to know that middle of last nite I woke up with itchy and intolerable coughing and awful stuffy throat—coughing and throat clearing again and again. Because I was soo tired, I just thought if I tough it out I could calm down and go back to sleep (too lazy to get up, turn on the lite to look for the tablets); but no luck—after consistently choking and coughing I forced myself up and put one tablet deep into the throat, as the tablet passed deeper into the throat, the coughing quieted down and I felt relaxed. Then the stuffy throat loosened up and I was able to get rid of the phlegm and then back to sleep within 10 min all the way until this morning!” In this subject, the loosening of phelgm was consistently observed and suggested that DAPA-2-5 may facilitate the reflexes for expectoration. It was noted by both subjects that the painful, hacking type of cough was attenuated by the DAPA-2-5 tablet, but the ability to expectorate the mucus was not impaired.

The second subject had cough variant asthma that was severely aggravated when he moved from the San Francisco Bay Area to Hong Kong. For three months, it was non-stop coughing and his social activities were curtailed. When introduced to the orally disintegrating tablets (ODT-A) containing 1.5 mg DAPA-2-5, he would take two or three tablets at a time to control his discomfort. He noted that the DAPA-2-5 ODT will take “the edge off his cough stimulus” in his throat. After a week's trial, his cough was completely under control and he remarked that “his life had been saved.”

In this subject, however, the deep-seated coughs originating from the trachea and bronchi were still irritating and painful. To modify delivery, DAPA-2-5, 4 mg/mL, was first dissolved in a solution of 25% wt/wt lemon juice and 1.5% wt/wt xylitol and the subject instructed to toss 1 mL of the solution (stored in a 2 mL microcentrifuge tube) into the back of his mouth. Surprisingly, this delivery system was more effective than the ODT. The subject felt as if the solution readily passed the upper esophageal sphincter and entered the esophagus to exert a robust cooling action. This cooling action was then felt to occur from the center of the chest and airways. Thus, a wider neuronal receptive field was activated.

Further modifications of the liquid delivery system were made by placing DAPA-2-5, as a 2 mg/mL solution (in distilled water) in a Boston Round ½ oz (14 mL) container, attached to a Yorker Spout (E.D. Luce packaging). The subject was instructed to place the tip of the Spout at the back of the mouth and gently squeeze out droplets. Approximately, 0.2 to 0.3 mL is delivered by this method. This was also an effective method control airway discomfort.

Case Study 5

A 67-year old liked to eat ice cream but had occasional “ice cream headaches”, a condition caused by reflex vasoconstriction of cerebral blood vessels in response to excessive cold. The individual was a chemist and scientist and upon hearing of the terms “dynamic cool” and “icy cold”, volunteered to try orally disintegrating tablets (ODT-A) each containing 2 mg of DAPA-2-5 or DAPA-2-7, to see if these substances would precipitate an “ice cream headache.” Neither of the ODT precipitated a headache response. The subject remarked on how the DAPA-2-5 ODT reminded him of the taste of obsolete syrups that contained chloroform. As a chemist, he was familiar with the taste of chloroform. He said that the DAPA-2-5 ODT had the same pleasant sweet taste, “organic” quality, and rapid onset of effect. This individual also liked to over-eat and had occasional bouts of regurgitation of stomach contents into the throat, precipitated by excess of pizza, ice cream, a capuccino, and a recumbent position. By rapid swallowing of two DAPA-2-5 ODT, he immediately controlled his throat discomfort from acid reflux and also the urge to vomit. On another occasion this individual had hiccups after swallowing food too fast. These hiccups were stopped within several min by taking two DAPA-2-5 ODT.

Case Study 6

A 78-year old Chinese female owned her own travel company and frequently led tour groups to Hong Kong, Shanghai, New York, and Los Angeles. She is energetic and healthy, but complained of hoarseness and “loss of voice” because of continual talking for long hours. She asked to try orally disintegrating tablets (ODT-A) containing 1 mg of DAPA-2-5. She said there was immediate relief from throat discomfort and she could again speak and communicate effectively. This beneficial effect has been repeatedly observed for twenty trials.

Case Study 7

Six women, all over the age of 60, suffered from sporadic throat discomfort over a period of more than 8 weeks. The causes of throat discomfort were allergies, excessive smoking, and psychogenic. Orally disintegrating tablets (ODT-A) containing 0 mg (placebo) or 1.0 mg of DAPA-2-5 were given to these individuals with the instructions of taking the ODTs on an “as needed basis” but not to exceed three tablets in any one day. These individuals were motivated to try the tablets to obtain sensory relief. The subjects had no difficulties in learning how to self-administer the ODT. The placebo ODT was immediately recognized as being not effective and rejected after one trial. The DAPA-2-5 ODT was 100% effective in reducing throat discomfort. The desired drug effect was achieved in all subjects.

The individuals not only felt better, but they stopped using all other medications stored in their medicine cabinets such as peppermint oil, antacids, Benadryl®, Mucinex®, and Chloraseptic®. There was no ambiguity about the ability of the DAPA-2-5 ODT to counteract pharyngeal irritation in all tested subjects.

Case Study 8

A 73-year old overweight male went to the golf driving range and hit a bucket of 100 balls and then proceeded to walk and play 18 holes. He was right-handed. Afterwards, he had a 5-course dinner with his friends and drank 3 glasses of wine.

Later in the evening, he complained of soreness and pain in his left pectoral muscle and supraclavicular region. Then he complained of tightness in the chest, pain behind the sternum, and shortness of breath. He felt an acid taste in his mouth and took some Alka-Seltzer, an antacid, and then a Zantac tablet. These medications did not relieve his chest pain or sense of malaise, and he felt anxious, flushed and sweaty. He worried that “the end might be near” and debated if he should call the Emergency Services at his hospital. He lived in the suburbs and so it was not convenient for him to drive into the city where his hospital was located.

He decided to try some experimental DAPA-2-5 tablets which had been given to him previously, and swallowed in one gulp (with water) three tablets each containing approximately 1.5 mg of DAPA-2-5. He said the sensation was that of cool water flooding his throat and percolating slowly inside his chest. The coolness was strong, but gradual and penetrating. The pain behind his sternum quickly diminished and he felt more comfortable and less agitated. He fell asleep and did not wake till the next morning. He then went to see his personal physician who measured his serum troponin levels and then put him through a cardiac stress test, using an exercise treadmill. His enzyme levels and electrocardiogram were both within normal limits. His physician advised him to watch his diet and weight, but otherwise not to worry about his heart which seemed to be healthy.

A 68-year old man went out with a group of friends to partake of Szechuan cuisine, where the specialty was a pork dish and a baked fish dish liberally cooked in Szechuan peppers and pepper oil. He drank MaoTai liquor [53% alcohol content by volume] during the course of the evening. Near midnight he started to experience chest pains, and broke out into a hot sweat. His face turned red and he gasped for breath. His colleagues wondered if he should be taken to the hospital. But then one of the guests, who smoked, remembered he had some DAPA-2-5 tablets in his pocket. He gave four of the tablets to the individual in distress who swallowed it with a gulp of water, and, to the surprise of everyone at the table, the symptoms of chest discomfort went away in about 10 min. The individual recovered and was able to drive himself back home.

In both cases, it seems that the symptoms of indigestion [non-cardiac pain] can be confused with chest pains caused by inadequate oxygenation of the heart muscle [e.g. angina or cardiac pain caused coronary arteries disorders]. The availability of a

DAPA-2-5 molecule to relieve non-cardiac pain, but not cardiac pain, would be a valuable adjunct to the differential diagnosis of the causes of chest pains.

Case Study 9

A 71-year old retired police officer was of muscular build but above ideal weight at 5 feet 5 inches (165 cm), and 185 lbs (84 kg). He had played soccer on his college team, had a short neck, and strong trapezius muscles. For at least five years he complained of poor sleep and daytime fatigue. Taking a sedative such as Ambien® did not help him sleep better and he was worried about impairment of his driving skills. His wife complained about his snoring and demanded to use a separate bedroom. Polysomnography tests indicated a borderline diagnosis of obstructive sleep apnea, but he could not tolerate using continuous positive airway pressure masks and machines because he said it gave him a sense of claustrophobia and suffocation. He volunteered to take tablets containing 2 mg DAPA-2-5 before going to sleep. His wife immediately noticed that he stopped snoring. He said that he slept better because the tablets gave him a refreshing sensation in the throat and a sense of relaxed breathing of cool air. He now uses the tablets on an “as needed basis.” He suggested that these tablets might also value in sleep apnea.

Overall Summary

Finding the right therapeutic molecule, among a group of similar congeners, is very much like looking for the proverbial needle in the haystack. The selection process depends on continued experiment.

The concept has been put forward that heat abstraction sensations, captured by topical application of a molecule, can be used to alleviate discomfort of non-keratinizng tissues. By synthesizing compounds and devising tests, a molecule name DAPA-2-5 was identified as having the selective desirable properties for achieving this purpose. DAPA-2-5 was selective for non-keratinizing stratified epithelia of the aerodigestive tract, producing a robust, cooling sensation, without irritation and sting. This effect is not seen in keratinized skin. On receptor targets, it was selective for TRPM8 and not TRPVI and TRPA1. When bioassayed on laboratory animals, DAPA-2-5 selectively inhibited heat edema and did not produce irritation on the skin. DAPA-2-5 was selectively the most potent analog in suppression of hydrochloric acid induced swallowing in the rat. When formulated into an ODT and tested on volunteers, DAPA-2-5 was selectively the best molecule for reducing sensory discomfort of the upper digestive tract.

1. A therapeutic method, comprising:
selecting a tissue site having a non-keratinizing stratified epithelial (NKSE) tissue thereon as causing sensory discomfort in a patient; and,
administering a therapeutically effective quantity of 1-di(sec-butyl)-phosphinoyl-pentane (DAPA-2-5) to the site.

2. The method as in claim 1 wherein the site is selected from the group consisting of of an upper aerodigestive tract surface, an oral cavity surface, a respiratory surface, a nasal membrane surface, an pharyngeal surface, an esophageal surface, and an anogenital surface.

3. The method as in claim 1 wherein the site is an upper aerodigestive tract surface.

4. The method as in claim 1 wherein the site is an oral cavity lining or an internal portion of the lips.

5. The method as in claim 1 wherein the site is a respiratory epithelial surface.

6. The method as in claim 1 wherein the site is a lumenal lining of a nasal membrane surface.

7. The method as in claim 1 wherein the site is a pharyngeal surface.

8. The method as in claim 1 wherein the sensory discomfort includes throat irritation.

9. The method as in claim 1 wherein the sensory discomfort includes laryngeal and esophageal discomfort.

10. The method as in claim 1 wherein the sensory discomfort includes heartburn and chest pain.

11. The method as in claim 1 wherein the sensory discomfort includes anogenital discomfort.

12. A method of selectively treating sensory discomfort of the upper digestive tract, comprising:
administering to a patient experiencing sensory discomfort a therapeutically effective amount of an active agent selected from the group consisting or 1-di(sec-butyl)-phosphinoyl-pentane (DAPA-2-5), 1-di(sec-butyl)-phosphinoyl-hexane, 1-di(sec-butyl)-phosphinoyl-heptane and combinations thereof.

13. The method as in claim 12 wherein the administering includes providing an orally disintegrating tablet in which the active agent is carried by a mineral excipient adapted to deliver the active agent onto membranes of the digestive tract when ingested.

14. The method as in claim 12 wherein the tablet has from 2 to 20 wt. % of the mineral excipient.

15. The method as in claim 12 wherein the tablet has from about 0.5 to 8 mg active agent.

16. An orally disintegrating tablet selectively useful in treating sensory discomfort from non-keratinizing stratified epithelial (NKSE) tissue, comprising:
from about 0.5 to 8 mg of an active agent, the active agent selected from the group consisting or 1-di(sec-butyl)-phosphinoyl-pentane (DAPA-2-5), 1-di(sec-butyl)-phosphinoyl-hexane, 1-di(sec-butyl)-phosphinoyl-heptane and combinations thereof; and,
from about 2 to 20% by weight of a mineral excipient.

17. The tablet as in claim 16 wherein the active agent consists essentially of 1-di(sec-butvI)-phosphinovl-pentane (DAPA-2-5).

18. The tablet as in claim 16 wherein said tablet weighs from about 40 to 150 mg.

19. The tablet as in claim 16 wherein the sensory discomfort includes cough or throat irritation.

20. The tablet as in claim 16 wherein the mineral excipient is adapted to deliver the compound onto upper membranes of the digestive tract.

21. The tablet as in claim 16 wherein the mineral excipient is magnesium aluminometasilicate represented by the empirical formula Al₂O₃.MgO.2SiO₂.xH₂O.

22. The tablet as in claim 16 wherein the mineral excipient is CaHPO₄, Spray Dried Granule, Dibasic Calcium Phosphate Anhydrous; Calcium Hydrogen Phosphate, Anhydrous.

23. A method of differential diagnosis of cardiac versus non-cardiac pain, comprising:
administering to a patient experiencing chest pains discomfort a therapeutically effective amount of an active agent selected from the group consisting or 1-di(sec-butyl)-phosphinoyl-pentane (DAPA-2-5), 1-di(sec-butyl)-phosphinoyl-hexane, or 1-di(sec-butyl)-phosphinoyl-heptane and combinations thereof; and, monitoring patient response to the administering.
